(19) United States
(12) Patent Application Publication
(10) Pub. No.: US 2014/0148499 A1
(43) Pub. Date: May 29, 2014

(54) METHODS AND REAGENTS FOR EFFICIENT CONTROL OF HIV PROGRESSION

(71) Applicants: Mireia Arnedo Valero, Sant Cugat del Valles (ES); Montserrat Plana Prades, Barcelona (ES); Josep Maria Gatell Artigas, E-Barcelona (ES)

(72) Inventors: Mireia Arnedo Valero, Sant Cugat del Valles (ES); Montserrat Plana Prades, Barcelona (ES); Josep Maria Gatell Artigas, E-Barcelona (ES)

(73) Assignees: FUNDACIÓ PRIVADA INSTITUT DE RECERCA DE LA SIDA, Badalona (ES); LABORATORIOS DEL DR. ESTEVE, S.A., Barcelona (ES)

(21) Appl. No.: 14/131,903

(22) PCT Filed: Jul. 12, 2012
(86) PCT No.: PCT/EP2012/063670

§ 371 Date: Feb. 4, 2014

Related U.S. Application Data
(60) Provisional application No. 61/508,348, filed on Jul. 15, 2011.

(30) Foreign Application Priority Data
Jul. 15, 2011 (EP) 11382243.1

(51) Int. Cl. C12N 15/113; A61K 31/713; A61K 45/06; C12Q 1/70

(52) U.S. Cl. CPC C12N 15/1132; C12Q 1/703; A61K 31/713; A61K 45/06
USPC 514/44 A; 435/5; 536/24.5; 536/24.32

(57) ABSTRACT

The invention relates to methods for the identification of HIV-1 infected subjects capable of controlling viral load based on the determination of the expression levels of several miRNAs. In addition, the invention also relates to methods for controlling HIV infection by using some of the differentially expressed miRNAs or polynucleotides encoding said miRNAs, as well as to compositions comprising a miRNA or a polynucleotide encoding said miRNA and an anti-HIV agent.

### FIELD OF THE INVENTION

The invention relates to the field of immunology and, in particular, to methods for the identification of patients capable of controlling HIV progression, as well as to the identification of specific miRNAs associated to HIV progression control and the uses thereof for improving the immunological response of HIV patients.

### BACKGROUND OF THE INVENTION

The Human Immunodeficiency Virus (HIV-1) infection is characterized either by an increased plasmatic viremia or by a progressive loss of CD4+ T lymphocytes. In the absence of antiretroviral therapy, this results in an important immunologic impairment with the consequent appearance of opportunistic infections and, lastly, death. In this regard, the highly active antiretroviral therapy (HAART) has produced dramatic changes in the progression and evolution of the HIV-1 infection. However, the inability of this treatment for eliminate the latent HIV-1 in the integrated proviral DNA, and the persistence of cryptic and low levels of viral replication, as well as other important concerns (i.e. toxicity, need of strict adherence to treatment), continue to be significant limitations to this therapy.

On the other hand, even if viral replication causes the progressive destruction of the immune system and the fatal evolution to AIDS in the majority of HIV-1 patients, there is a low proportion of individuals immunologically “privileged”, the long term non-progressors (LTNPs), who maintain a stable CD4 T cell counts. LTNPs are also characterized by a considerable anti-HIV-1 T cell immune response (Th, CTL) that is conserved along time and the presence of neutralizing antibodies. These responses are associated with the control of viral replication and are related to a very low or undetectable viral load in plasma that cannot be attributed to a defective virus, protective genetic variants of the HIV-1 co-receptors or their regulating chemokines. Within the LTNP group, less than 1% of the subjects are also able to maintain a viral load in plasma below detectable limits (i.e. <50 copies/mL). These individuals are referred to as elite controllers (ECs). See Lambote O, et al., Clin. Infect. Dis. 2005; 41:1053-1056 and Grabar S, et al., AIDS 2009; 23:1163-1169. This group of patients is important for defining the parameters involved in the control of viral replication, and thus, its study could be helpful for the future development of effective treatments and vaccines. The viremic control exercised by the host could be due many causes such as viral, immunological, or genetic factors or a combination thereof.

Several studies have tried to dissect the factors involved in viremic control. These attempts have focused mostly in the analysis of the differences in antiviral immune responses between EC individuals. In this sense, it has been described that the presence of an earlier and effective innate immune response could be important. More precisely, ECs seem to maintain both the normal phenotype of NK cells and the preserved cytotoxic capacity of these cells. It is not known if this feature is relevant for the control of viral replication, but it has been proposed that NK cells could act in conjunction with dendritic cells early during the adaptive immune response and in the modulation of the inflammatory phenomenon observed during the HIV-1 pathogenic infection. Similar to natural hosts infected with SIV, ECs have a lower depletion of CD4+ T cells in mucosa and a lower level of bacterial translocation than normal progressors. This fact could be partially related to a better type I production of IFN and other inflammatory cytokines by plasmacytoid dendritic cells, which may induce a chronic activation of the immune system at a moderate level.

Concerning humoral immunity, ECs do not show greater titers of broad range neutralizing antibodies than chronic progressors, suggesting that viremic control could not be attributed to the protective role of this arm of the immune response. In contrast, the most relevant findings are related to the role of the cellular immune response in the control of the HIV-1 infection. In this regard, ECs present a large number of HIV-1-specific CD8+ T cells. These cells are polyfunctional (e.g. production of IFN-γ, TNF-α, IL-2, MIP-1(3; cytotoxic activity, proliferative capacity) in the peripheric tissue and mucosa. It has been shown that the greater production of cytolytic enzymes (e.g. granzyme B, perforin) reduces viral replication in autologous CD4+ T cells in ECs in contrast with HIV-+ patients HAART-treated and with suppressed viremia.

The importance of the response of CD8+ T cells in ECs is suggested also by the overexpression of certain HLA class I alleles described as protectors, such as HLA B*57 and B*27. Despite this evidence, CTL response is not sufficient to explain the control of viral replication in all ECs, since there is some heterogeneity within this group. ECs have shown also an increased number of anti-HIV+ CD4+ T cells with enhanced proliferative capacity. This feature is either dependent on the IL-2 level or the reduction of the pool of exhausted cells, since the inhibitory molecule CTLA-4 is not overexpressed in the EC's HIV-specific CD4 T cells, in contrast to HIV progressors. Finally, the existence of a balance between the protective antiviral immune response of CD4 and CD8 T cells and the modulation mechanisms exerted by regulatory T cells (Tregs) could also play a role in viral replication. This could minimize the nonspecific and undesirable activation phenomena. In this sense, it has been described that the presence of high levels of Tregs in ECs correlates with a low level of CD4+ T cell activation.

A better understanding of the causes of the differences in VL could provide relevant pointers in the development of new vaccines and drugs for controlling HIV.

### SUMMARY OF THE INVENTION

In a first aspect, the invention relates to a method for the identification of a controller HIV-1 infected subject which comprises determining the level of one or more miRNAs selected from the group consisting of miR-27a, miR-27b, miR-29b, miR-221 miR-155, miR-146a, miR-484, miR-339, miR-186, miR-106a, miR-17, miR-590, miR-374b, miR-140-Sp, miR-16, miR-454, miR-200c, miR-146b, miR-200b, miR-422a, miR-125a-Sp, miR-191 and miR-197 in a sample from said subject, wherein an increased expression level of one or more of the miR-27a, miR-27b, miR-29b or miR-221 miRNAs with respect to a reference value is indicative that the subject is a HIV controller, or wherein a decreased expression level of one or more of miR-155, miR-146a, miR-484, miR-339, miR-186, miR-106a, miR-17, miR-590, miR-374b, miR-140-Sp, miR-16, miR-454, miR-200c, miR-146b, miR-200b, miR-422a, miR-125a-5p, miR-191 or miR-197 with respect to a reference value is indicative that the subject is a HIV controller.

In a second aspect, the invention relates to a miRNAs selected from the group consisting of miR-27a, miR-27b, miR-29b, miR-125, miR-146, miR-155 and miR-221 for use in the treatment or prevention of a disease caused by HIV.

In a third aspect, the invention relates to a microbicide composition or kit-of-parts comprising a miRNA selected from the group consisting of miR-27a, miR-27b, miR-29b, miR-125, miR-146, miR-155 and miR-221 or a polynucleotide encoding said miRNA. Preferably, the composition or kit-of-parts comprises also an anti-HIV agent.

In a fourth aspect, the invention relates to a kit comprising reagents adequate for the determination of the expression levels of one or more miRNAs selected from the group consisting of miR-27a, miR-27b, miR-29b, and miR-221.

### DESCRIPTION OF THE FIGURES

FIG. 1. Study groups: miRNA isolated from 8 million of PBMCs activated with PHA were compared between these groups: 1) elite controllers (HIV patients with VL<200 cp/mL without treatment); 2) viremic progressors (HIV patients with VL>5000 cp/mL without treatment); 3) ART (HIV patients with VL<200 cp/mL during HAART); 4) HIV negative

FIG. 2. Hierarchical clustering of differentially expressed miRNAs between elite controllers, viremic progressors and ART patients.

FIG. 3. Mean fold change EC and VP versus HIV negative. The figure shows the second order fold change (−ΔCt) in the 23 miRNAs most differentially expressed between: i) elite controllers and viremic progressors and ii) HIV negative subject. The dotted line indicates a 2-fold difference in expression compared with the mean of expression for all PBMCs.

FIG. 4. Relative expression of 22 miRNAs of elite controllers and ART patients compared to HIV negative subjects.

FIG. 5. Relative expression of 22 miRNAs of elite controllers and ART patients compared to viremic progressors.

FIG. 6. Validation results for miR-221, miR-29b, miR125a, and miR146a in elite controllers, viremic controllers, viremic progressors and ART patients.

### DETAILED DESCRIPTION OF THE INVENTION

### 1. Definitions of General Terms and Expressions

The term “AIDS”, as used herein, refers to the symptomatic phase of HIV infection, and includes both Acquired Immune Deficiency Syndrome (commonly known as AIDS) and “ARC,” or AIDS-Related Complex. See Adler M, et al., Brit. Med. J. 1987; 294: 1145-1147. The immunological and clinical manifestations of AIDS are known in the art and include, for example, opportunistic infections and cancers resulting from immune deficiency.

The terms “anti-HIV agent”, “HIV-inhibiting agent” and “HIV antiviral agent”, as used herein, refer to any compound or a pharmaceutically acceptable salt thereof capable of inhibiting the replication of HIV in a cell (e.g. mammalian cell) or effective in treating, preventing, or delaying the onset or progression of HIV infection or AIDS, or diseases or conditions arising therefrom or associated therewith.

The term “antiretroviral therapy” or “ART”, as used herein, refers to the administration of one or more antiretroviral drugs to inhibit the replication of HIV. Typically, ART involves the administration of at least one antiretroviral agent (or, commonly, a cocktail of antiretrovirals) such as a nucleoside reverse transcriptase inhibitor (e.g. zidovudine, AZT, lamivudine (3TC) and abacavir), a non-nucleoside reverse transcriptase inhibitor (e.g. nevirapine and efavirenz), or a protease inhibitor (e.g. indinavir, ritonavir and lopinavir). The term Highly Active Antiretroviral Therapy (“HAART”) refers to treatment regimens designed to aggressively suppress viral replication and progress of HIV disease, usually consisting of three or more different drugs such as, for example, two nucleoside reverse transcriptase inhibitors and a protease inhibitor.

The term “B cell”, as used herein, refers to any member of a diverse population of morphologically similar cell types that develop in the bone marrow and that mediate the humoral immune response of the adaptive immune system. B cells are characterized by the presence of a B-cell receptor able to bind specifically to an antigen. Their principal functions are to make antibodies against antigens, perform the role of antigen-presenting cells (APCs) and eventually develop into memory B cells after activation by antigen interaction. See Alberts B, et al., Molecular Biology of the Cell (Garland Publishing Inc., New York, N.Y., US, 2008, pp. 1363-1391).

The term “CD4+ T cells”, as used herein, refers to a type of T cells that expresses the CD4 marker. Said CD4+ T cells are generally treated as having a pre-defined role as helper T cells within the immune system. “CD4”, as used herein, refers to a cluster of differentiation 4, a glycoprotein expressed on the surface of T helper cells, monocytes, macrophages, and dendritic cells. CD4 is a co-receptor that assists the T cell receptor (TCR) with an antigen-presenting cell. Using its portion that resides inside the T cell, CD4 amplifies the signal generated by the TCR by recruiting an enzyme, known as the tyrosine kinase lck, which is essential for activating many molecules involved in the signaling cascade of an activated T cell. The complete protein sequence for human CD4 has the UniProt accession number P01730 (Jun. 18, 2012).

The term “CD8+ T cells”, as used herein, refers to a type of T cell that expresses the CD8 marker. “CD8”, as used herein, refers to cluster of differentiation 8, a transmembrane glycoprotein that serves as a co-receptor for the T cell receptor (TCR) expressed in the cytotoxic T cells implicated in the rejection of transplants and the destruction of tumor and virally infected cells. The complete protein sequence for human CD8 has the UniProt accession number P10966 (Jun. 18, 2012).

The term “combined administration”, as used herein, means that the compounds which form part of a composition or kit-of-parts according to the invention may be administered jointly or separately, simultaneously, at the same time or sequentially in the treatment of the pathologies previously mentioned in any order. For example, the administration of the miRNA or the polynucleotide encoding a miRNA may be performed first, followed by the administration of one or more therapeutic agents useful in the treatment of diseases caused by HIV infection; or the administration of the miRNA or the polynucleotide encoding a miRNA may be performed at the end, preceded by the administration of one or more therapeutic agents useful in the treatment of diseases caused by HIV infection; or the administration of the miRNA or the polynucleotide encoding a miRNA may be performed at the same time as the administration of one or more therapeutic agents useful in the treatment diseases caused by HIV infection.

The term “comprising” or “comprises”, as used herein, discloses also “consisting of” according to the generally accepted patent practice.

The term “controller”, as used herein, refers to a HIV infected subject that exhibits a decrease in HIV viral load after infection and that maintains said decreased viral load levels over time. A “controller” also refers to an HIV-1 infected subject who remains asymptomatic with normal CD4+ T-cell counts and low or undetectable plasma viral loads despite having never been treated with antiretroviral medications. HIV controllers are capable of maintaining very low viral load levels, for example, plasma HIV RNA levels <2000 copies/mL in the absence of antiretroviral therapy, measured three times over a period spanning at least 12 months. The features of controllers as defined by the HIV Controller Consortium (http://www.hivcontrollers.org/, July 2012) are: i) to maintain HIV RNA levels below 2000 copies/mL, ii) no antiretroviral therapy for 1 year or longer and iii) episodes of viremia are acceptable as long as they represent the minority of all available determinations.

The term “decreased”, as used herein, refers to the level of miRNA of a subject at least 1-fold (e.g. 1, 2, 3, 4, 5, 10, 20, 30, 40, 50, 60, 70, 80, 90, 100, 1000, 10,000-fold or more) lower than its reference value. “Decreased”, as it refers to the level of miRNA of a subject, signifies also at least 5% lower (e.g. 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100%) than the level in the reference sample or with respect to the reference value for said miRNA.

The term “disease caused by HIV-1 infection”, as used herein, refers to any disease which results as a consequence of a HIV infection. Most preferably, the disease is selected from the group consisting of Acquired Immune Deficiency Syndrome (AIDS) or the HIV- or HAART-associated disorders HIV-associated dementia (HAD), Immune Reconstitution Disease (IRD) and lipodystrophy.

The term “elite controller”, as used herein, refers to a HIV infected subject that shows HIV RNA below its level of detection in the absence of antiviral therapy for the respective available ultrasensitive assay (e.g. <75 copies per mL by bDNA or <50 copies per mL by ultrasensitive PCR). Criteria for identifying elite controllers have been described previously. See Goudsmit J, et al., AIDS 2002; 16:791-793. The features of controllers as defined by the HIV Controller Consortium (http://www.hivcontrollers.org/, July 2012) are: i) to maintain HIV RNA levels below 50 copies/mL, ii) no antiretroviral therapy for 1 year or longer, and iii) episodes of viremia are acceptable as long as there are not consecutive episodes

The term “HIV”, as used herein, include HIV-1 and HIV-2 and SIV. “HIV-1” means the human immunodeficiency virus type-1. HIV-1 includes, but is not limited to, extracellular virus particles and the forms of HIV-1 associated with HIV-1 infected cells. HIV-1 is known to comprise at least ten subtypes (A1, A2, A3, A4, B, C, D, E, PL F2, G, H, J and K). See Taylor B, et al., MEJM 2008; 359(18): 1965-1966. Subtype B has been associated with the HIV epidemic in homosexual men and intravenous drug users worldwide. Most HIV-1 immunogens, laboratory adapted isolates, reagents and mapped epitopes belong to subtype B. In sub-Saharan Africa, India, and China, areas where the incidence of new HIV infections is high, HIV-1 subtype B accounts for only a small minority of infections, and subtype HIV-1 C appears to be the most common infecting subtype. “HIV-2” means the human immunodeficiency virus type-2. HIV-2 includes, but is not limited to, extracellular virus particles and the forms of HIV-2 associated with HIV-2 infected cells. HIV-2 is known to include at least five subtypes (A, B, C, D, and E). The term “SIV” refers to simian immunodeficiency virus which is an HIV-like virus that infects monkeys, chimpanzees, and other nonhuman primates. SIV includes, but is not limited, to extracellular virus particles and the forms of SIV associated with SIV infected cells.

The term “HIV immunogen”, as used herein, refers to a protein or peptide antigen derived from HIV capable of generating an immune response in a subject. HIV immunogens for use according to the present invention may be selected from any HIV isolate (e.g. any primary or cultured HIV-1, HIV-2, or HIV-3 isolate, strain, or Glade). Thus, in certain embodiments, it may be preferable to select immunogens from particular subtypes (e.g. HIV-1 subtypes B or C), it may be desirable to include immunogens from multiple HIV subtypes (e.g. HIV-1 subtypes B and C HIV-2 subtypes A and B, or a combination of HIV-1, HIV-2, or HIV-3 subtypes) in a single immunological composition.

The term “identification”, as used herein, refers to methods known in the art for estimating and even determining whether or not a subject is suffering from a given disease or condition (e.g. whether the subject is a HIV controller). A diagnosis is often made on the basis of one or more indicators such as, for example, a biomarker (e.g. a miRNA or a set of miRNAs), the amount (including presence or absence) of which is indicative of the presence, severity, or absence of the condition. It also relates to evaluating the probability according to which a subject suffers from a disease. Such evaluation may not be correct for 100 percent of the subjects to be diagnosed. The term, however, requires that a statistically significant part of the subjects can be identified as suffering from the disease or having a predisposition for it. There are several statistic evaluation tools (e.g. p value, Student's t-test, Mann-Whitney test) known in the art for analyzing this probability. See Dowdy S, et al., Statistics for Research (John Wiley and Sons, New York, N.Y., US, 1983). The preferred confidence intervals are of at least 50%, at least 60%, at least 70%, at least 80%, at least 90%, or at least 95%. The p values are preferably 0.2, 0.1, or 0.05.

The term “immunogenic composition”, as used herein, refers to a composition that elicits an immune response which produces antibodies or cell-mediated immune responses against a specific immunogen. As used herein, “immune response” refers to a response made by the immune system of an organism to a substance, which includes but is not limited to foreign or self proteins. Particularly, “immune response” refers to a CD8+ T-cell mediated immune response to HIV infection. There are three general types of “immune response” including, but not limited to, mucosal, humoral, and cellular “immune responses.”

The term “increased”, as used herein, refers to the level of miRNA of a subject at least 1-fold (e.g. 1, 2, 3, 4, 5, 10, 20, 30, 40, 50, 60, 70, 80, 90, 100, 1000, 10,000-fold or more) greater than its reference value. “Increased”, as it refers to the level of miRNA of a subject, signifies also at least 5% greater (e.g. 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100%) than the level in the reference sample or with respect to the reference value for said miRNA.

The term “kit”, as used herein, refers to a product containing the different reagents necessary for carrying out the methods of the invention packed so as to allow their transport and storage. Materials suitable for packing the components of the kit include crystal, plastic (e.g. polyethylene, polypropylene, polycarbonate), bottles, vials, paper, or envelopes. The term “kit-of-parts”, as used herein, shall encompass an entity of physically separated components, which are intended for individual use, but in functional relation to each other.

The term “microbicide”, as used herein, refers to a compound capable of killing, inhibiting the growth of, or controlling the growth of, HIV. The microbicide compositions according to the invention are useful for the treatment or prevention of diseases caused by HIV infection.

The term “miRNA”, also known as microRNA, as used herein, refers to double-stranded RNA molecules, typically of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Following processing, mature miRNAs are incorporated into a RISC(RNA-Induced Silencing Complex), which participates in RNA interference (RNAi). miRNAs can pair with target mRNAs that contain sequences only partially complementary (e.g. 30%, 35%, 40%, 50%, 55%, 60%, 65%, 70%, 75%, 80%, or more) to the miRNA. Such pairing results in repression of mRNA translation without altering mRNA stability. Alternatively, miRNAs with a substantial degree of complementarity to their targets effect gene silencing by mediating mRNA degradation. See Hutvagner G, et al., Science 2002; 297:2056-2060. As expression of precursor microRNAs (i.e. pri-miRNAs) is often developmentally regulated, miRNAs are often referred to interchangeably in the art as “small temporal RNAs” or “stRNAs”.

The terms “miR-27a”, “miR-27b”, “miR-29b”, “miR-221”, “miR-155”, “miR-146a”, “miR-484”, “miR-339”, “miR-186”, “miR-106a”, “miR-17”, “miR-590”, “miR-374b”, “miR-140-Sp”, “miR-16”, “miR-454”, “miR-200c”, “miR-146b”, “miR-200b”, “miR-422a”, “miR-125a-Sp”, “miR-191” and “miR-197”, as used herein, refer to the human miRNAs as defined in the miRBase::Sequences database of the Wellcome Trust Sanger Institute (http://microrna.sanger.ac.uk/sequences/index.shtml July 2012). The nucleic acid sequences corresponding to the miRNAs, hairpin pre-miRNAs and miRNA* s described in Tables 1-3 are available from the miRBase::Sequences database of the Wellcome Trust S anger Institute (http://microma.sanger.ac.uk/sequences/index.shtml July 2012). miRNA, hairpin miRNA, and miRNA* sequence information can be found in Release 17 (April 2011). See Griffiths-Jones S, et al., Nucleic Acids Res. 2008; 36 (Database Issue):D154-D158; Griffiths-Jones S, et al., Nucleic Acids Res. 2006; 34 (Database Issue):D140-D144 and Griffiths-Jones S, et al., Nucleic Acids Res. 2004; 32 (Database Issue):D109-D111. Nomenclature conventions used to describe miRNA sequences are described further in Ambros V, et al., RNA 2003; 9:277-279.

The term “operably linked”, as used herein, is intended to mean that the nucleotide sequence of interest is linked to a regulatory sequence(s) in a manner allowing the expression of the nucleotide sequence (e.g. in an in vitro transcription/translation system or in a host cell when the vector is introduced into the host cell). See Auer H, Nature Biotechnol. 2006; 24: 41-43. “Regulatory sequences” include promoters, enhancers, and other expression control elements (e.g. polyadenylation signals). See Goeddel D, Gene Expression Technology: Methods in Enzymology (Academic Press, Inc., San Diego, Calif., US, 1990). Regulatory sequences include those that direct constitutive expression of a nucleotide sequence in many types of host cells and those that direct expression of nucleotide sequences in certain host cells only (e.g. tissue-specific regulatory sequences). The design of the expression cassette can depend on several factors such as the choice of host cell, the level of expression of the miRNA or precursor thereof. The expression cassettes typically include one or more appropriately positioned sites for restriction enzymes, to facilitate the introduction of the nucleic acid into a vector.

The terms “pharmaceutically acceptable carrier,” “pharmaceutically acceptable diluent,” or “pharmaceutically acceptable excipient”, or “pharmaceutically acceptable vehicle”, as used interchangeably herein, refer to a non-toxic solid, semisolid or liquid filler, diluent, encapsulating material or formulation auxiliary of any conventional type. A pharmaceutically acceptable carrier is essentially non-toxic to recipients at the dosages and concentrations employed, and is compatible with other ingredients of the formulation. For example, the carrier for a formulation containing polypeptides would not normally include oxidizing agents and other compounds that are known to be deleterious to polypeptides. Suitable carriers include, but are not limited to, water, dextrose, glycerol, saline, ethanol, and combinations thereof. The carrier can contain additional agents such as wetting or emulsifying agents, pH buffering agents, or adjuvants which enhance the effectiveness of the formulation. Adjuvants could for example be selected from the group consisting of: AlK(SO₄)₂, AlNa(SO₄)₂, AlNH₄(SO₄), silica, alum, Al(OH)₃, Ca₃(PO₄)₂, kaolin, carbon, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-sn-glycero-3-hydroxphosphoryloxy)-ethylamine (CGP 19835A, also referred to as MTP-PE), RIBI (MPL+TDM+CWS) in a 2% squalene/Tween-80®emulsion, lipopolysaccharides and its various derivatives, including lipid A, Freund's Complete Adjuvant (FCA), Freund's Incomplete Adjuvants, Merck Adjuvant 65, polynucleotides (e.g. poly IC and poly AU acids), wax D from mycobacterium, tuberculosis, substances found in Corynebacterium parvum, Bordetella pertussis, and members of the genus brucella, Titermax, ISCOMS, Quil A, ALUN, Lipid A derivatives, choleratoxin derivatives, HSP derivatives, LPS derivatives, synthetic peptide matrixes or GMDP, interleukin 1, interleukin 2, Montanide ISA-51 and QS-21, CpG oligonucleotide, poly I:C and GM-CSF. See Hunter R, U.S. Pat. No. 5,554,372, and Jager E, et al., WO1997028816.

The term “polynucleotide encoding a miRNA”, as used herein, refers to polynucleotide comprising a mature sequence of one or more miRNAs. The polynucleotide may comprise the sequence encoding the pri-miRNA or pre-miRNA sequence for the one or more miRNAs. The polynucleotide comprising the mature, pre-miRNA, or pri-miRNA sequence can be single stranded or double stranded. The polynucleotides may contain one or more chemical modifications (e.g. locked nucleic acids, peptide nucleic acids, sugar modifications (e.g. 2′-O-methyl, 2′-O-methoxyethyl), 2′-fluoro, and 4′ thio modifications, and backbone modifications (e.g. one or more phosphorothioate, morpholino, or phosphonocarboxylate linkages).

The terms “prevent,” “preventing,” and “prevention”, as used herein, refer to inhibiting the inception or decreasing the occurrence of a disease in an animal. Prevention may be complete (e.g. the total absence of pathological cells in a subject). The prevention may also be partial, such that for example the occurrence of pathological cells in a subject is less than that which would have occurred without the present invention. Prevention also refers to reduced susceptibility to a clinical condition.

The term “protease inhibitor”, as used herein, refers to inhibitors of the HIV-1 protease, an enzyme required for the proteolytic cleavage of viral polyprotein precursors (e.g. viral gag and gag pol polyproteins) into the individual functional proteins found in infectious HIV-1. Suitable protease inhibitors that can be combined with the miRNAs or polynucleotides encoding miRNAs according to the invention is selected from the group consisting of ritonavir, lopinavir, saquinavir, amprenavir, fosamprenavir, nelfmavir, tipranavir, indinavir, atazanavir, TMC-126, darunavir, mozenavir (DMP-450), JE-2147 (AG1776), L-756423, KNI-272, DPC-681, DPC-684, telinavir (SC-52151), BMS 186318, droxinavir (SC-55389a), DMP-323, KNI-227, 1-[(2-hydroxyethoxy)methyl]-6-(phenylthio)-thymine, AG-1859, RO-033-4649, R-944, DMP-850, DMP-851, and brecanavir (GW640385).

The expression “reagent which allows determining the expression level of a gene”, as used herein, means a compound or set of compounds that allows determining the expression level of a gene either by means of the determination of the level of mRNA or by means of the determination of the level of protein.

The term “reference value”, as used herein, refers to the expression level of a miRNA under consideration in a reference sample. A “reference sample”, as used herein, means a sample obtained from subjects, preferably two or more subjects, known to be free of the disease or, alternatively, from the general population. The suitable reference expression levels of miRNAs can be determined by measuring the expression levels of said miRNAs in several suitable subjects, and such reference levels can be adjusted to specific subject populations. The reference value or reference level can be an absolute value; a relative value; a value that has an upper or a lower limit; a range of values; an average value; a median value, a mean value, or a value as compared to a particular control or baseline value. A reference value can be based on an individual sample value such as, for example, a value obtained from a sample from the subject being tested, but at an earlier point in time. The reference value can be based on a large number of samples, such as from population of subjects of the chronological age matched group, or based on a pool of samples including or excluding the sample to be tested.

The term “reverse transcriptase inhibitors”, as used herein, refers to any compound which inhibits the activity of HIV-1 reverse transcriptase, the enzyme which catalyzes the conversion of viral genomic HIV-1 RNA into proviral HIV-1 DNA.

The term “sample”, as used herein, refers to a small part of a subject, representative of the whole and may be constituted by tissue or a body fluid. Biopsies are small pieces of tissue and may be fresh, frozen or fixed, such as formalin-fixed and paraffin embedded (FFPE). Body fluid samples may be blood, plasma, serum, urine, sputum, cerebrospinal fluid, milk, or ductal fluid samples and may likewise be fresh, frozen or fixed. Samples may be removed surgically, by extraction (i.e. by hypodermic or other types of needles), by microdissection or laser capture. The sample should contain any biological material suitable for detecting the desired biomarker (miRNA), thus, said sample should, advantageously comprise cell material from the subject. Examples of the biological sample include whole blood, PBMC, and T cells. Methods for collecting and preparing these biological samples are known in the art.

The term “subject”, as used herein, refers to an individual, plant or animal, such as a human, a non-human primate (e g chimpanzees and other apes and monkey species); farm animals, such as birds, fish, cattle, sheep, pigs, goats and horses; domestic mammals, such as dogs and cats; laboratory animals including rodents, such as mice, rats and guinea pigs. The term does not denote a particular age or sex. The term “subject” encompasses an embryo and a fetus.

The term “T cell”, as used herein, refers to any member of a diverse population of morphologically similar lymphocytes types that develop in the thymus and that mediate the cellular immune response of the adaptive immune system. They are characterized by the presence of a T cell receptor on the cell surface. There are several subsets of T cells, each with a distinct function (i.e. helper, memory, regulatory, natural killer). See Alberts, 2008, supra, pp. 1364-1374, 1392-1409.

The term “treat” or “treatment”, as used herein, refers to controlling the progression of a disease before or after clinical signs have appeared. Control of the disease progression is understood to mean the beneficial or desired clinical results that include, but are not limited to, reducing the symptoms, reducing the duration of the disease, stabilizing a pathological state (specifically to avoid additional deterioration), delaying the progression of the disease, improving the pathological state and disease remission (both partial and total). The control of progression of the disease also involves an extension of the actual survival period compared to the expected survival period of a subject if treatment is withheld.

The term “Treg” or “regulatory T cell”, as used herein, refers to a T cell that expresses at least the CD4 or CD25 marker and which is capable of reducing or suppressing the activity of a T cell. This term includes T cells producing low levels of IL-2, IL-4, IL-5, and IL-1, and which suppress the activation of the immune system. Regulatory T cells suppress actively the proliferation and cytokine production of TH₁, TH₂, or naïve T cells which have been stimulated in culture with an activating signal (e.g. antigen and antigen presenting cells or with a signal that mimics antigen in the context of HLA such as, for instance, an anti-CD3 antibody plus an anti-CD28 antibody). Treg cells may express the FoxP3 marker.

The term “vaginal cream”, as used herein, refers to a semi-solid preparation suitable for application to the vaginal tract. Various classes of excipients or vehicles known in the art can be used in preparing vaginal creams. The excipients comprise materials of naturally occurring or synthetic origin that do not affect adversely the components of the formulation. Suitable carriers for use in the preparation of a vaginal cream include, but are not limited to, purified water, white soft paraffin, mucoadhesive polymers, liquid paraffin, polysorbate 60, sorbitan stearate silicone, waxes, petroleum, jelly, polyethylene glycol, and a variety of other materials, depending on the specific type of formulation used.

The term “viral entry inhibitor”, as used herein, refers to any compound capable of interfering with the entry of viruses into cells.

1. Method for the Identification of HIV Controller Patients

The present invention is based on the surprising finding that the expression levels of certain selected miRNAs vary in HIV-1 elite controllers, viremic progressors and viremic controllers. Thus, in a first aspect, the invention relates to a method for the identification of a HIV-1 controller subject which comprises determining the level of one or more miRNAs selected from the group consisting of miR-27a, miR-27b, miR-29b, miR-221 miR-155, miR-146a, miR-484, miR-339, miR-186, miR-106a, miR-17, miR-590, miR-374b, miR-140-Sp, miR-16, miR-454, miR-200c, miR-146b, miR-200b, miR-422a, miR-125a-Sp, miR-191 and miR-197 in a sample from said subject, wherein an increased expression level of one or more of the miR-27a, miR-27b, miR-29b or miR-221 miRNAs with respect to a reference value is indicative that the subject is a HIV controller, or wherein a decreased expression level of one or more of miR-155, miR-146a, miR-484, miR-339, miR-186, miR-106a, miR-17, miR-590, miR-374b, miR-140-Sp, miR-16, miR-454, miR-200c, miR-146b, miR-200b, miR-422a, miR-125a-5p, miR-191 or miR-197 with respect to a reference value is indicative that the subject is a HIV controller. In a preferred embodiment, the controller is an elite controller. In another preferred embodiment, the controller is a viremic controller.

In a first step, the method for the identification of HIV controller subject according to the present invention comprises determining the level of one or more of a miRNA selected from the group consisting of miR-27a, miR-27b, miR-29b, miR-221 miR-155, miR-146a, miR-484, miR-339, miR-186, miR-106a, miR-17, miR-590, miR-374b, miR-140-Sp, miR-16, miR-454, miR-200c, miR-146b, miR-200b, miR-422a, miR-125a-Sp, miR-191 and miR-197 in a sample from said subject.

TABLE 1
miRNA comparison EC with VP

| | | Adj. P (FDR)* | | SEQ |
|---|---|---|---|---|
| | | (Benjamini/ | | ID |
| Detector-miRNA | p-value | Hochberg) | Mature Sequence | NO: |
| Hsa-miR-454 | 7.78E−04 | 0.034755975 | UAGUGCAAUAUUGCU | 1 |
| | | | UAUAGGGU | |
| Hsa-miR-590-5p | 0.0001131326 | 0.034755975 | GAGCUUAUUCAUAAA | 2 |
| | | | AGUGCAG | |
| Hsa-miR-146b-5p | 0.001629186 | 0.034755975 | UGAGAACUGAAUUCC | 3 |
| | | | AUAGGCU | |
| Hsa-miR-200c | 0.001629186 | 0.034755975 | UAAUACUGCCGGGUA | 4 |
| | | | AUGAUGGA | |
| Hsa-miR-422a | 0.001629186 | 0.034755975 | ACUGGACUUAGGGUC | 5 |
| | | | AGAAGGC | |
| Hsa-miR-27b | 0.001629186 | 0.034755975 | UUCACAGUGGCUAAG | 6 |
| | | | UUCUGC | |
| Hsa-miR-27a | 0.002322095 | 0.037153512 | UUCACAGUGGCUAAG | 7 |
| | | | UUCUGC | |
| Hsa-miR-16 | 0.002322095 | 0.037153512 | UAGCAGCACGUAAAU | 8 |
| | | | AUUGGCG | |
| Hsa-miR-221 | 0.003275898 | 0.0044963185 | AGCUACAUUGUCUGC | 9 |
| | | | UGGGUUUC | |
| Hsa-miR-191 | 0.00457444 | 0.0044963185 | CAACGGAAUCCCAAA | 10 |
| | | | AGCAGCUG | |
| Hsa-miR-374b | 0.00457444 | 0.0044963185 | AUAUAAUACAACCUG | 11 |
| | | | CUAAGUG | |
| Hsa-miR-29b | 0.00457444 | 0.0044963185 | UAGCACCAUUUGAAA | 12 |
| | | | UCAGUGUU | |
| Hsa-miR-339-3p | 0.00457444 | 0.0044963185 | UCCCUGUCCUCCAGG | 13 |
| | | | AGCUCACG | |
| Hsa-miR-146a | 0.006322948 | 0.0044963185 | UGAGAACUGAAUUCC | 14 |
| | | | AUGGGUU | |
| Hsa-miR-17 | 0.006322948 | 0.0044963185 | CAAAGUGCUUACAGU | 15 |
| | | | GCAGGUAG | |
| Hsa-miR-125a-5p | 0.006322948 | 0.0044963185 | UCCCUGAGACCCUUU | 16 |
| | | | AACCUGUGA | |
| Hsa-miR-106a | 0.006322948 | 0.0044963185 | AAAAGUGCUUACAGU | 17 |
| | | | GCAGGUAG | |
| Hsa-miR-200b | 0.006322948 | 0.04814771 | UAAUACUGCCUGGUA | 18 |
| | | | AUGAUGA | |
| Hsa-miR-484 | 0.008651542 | 0.04814771 | UCAGGCUCAGUCCCC | 19 |
| | | | UCCCGAU | |
| Hsa-miR-155 | 0.008651542 | 0.04814771 | UUAAUGCUAAUCGUG | 20 |
| | | | AUAGGGGU | |
| Hsa-miR-140-5p | 0.008651542 | 0.04814771 | CAGUGGUUUUACCCU | 21 |
| | | | AUGGUAG | |
| Hsa-miR-197 | 0.008651542 | 0.04814771 | UUCACCACCUUCUCC | 22 |
| | | | ACCCAGC | |
| Hsa-miR-186 | 0.008651542 | 0.04814771 | CAAAGAAUUCUCCUU | 23 |
| | | | UUGGGCU | |

5% of false discovery range (FDR)

The method of the invention requires determining miRNA expression levels in a cell or in a biological sample. Methods for determining miRNA expression levels in cells or biological samples include generic methods for the detection and quantification of nucleic acids, especially RNA, and optimized methods for the detection and quantification of small RNA species. Illustrative, non-limitative, examples of methods which can be used to facilitate the testing of the multiple miRNAs of the invention include:
1) methods based in hybridization reactions, such as Northern blot analysis and in situ hybridization,
2) multiplex or singleplex real-time RT-PCR (reagents available from, e.g. Applied Biosystems, Inc., Foster City Calif., US and System Biosciences, Inc. (SBI), Mountain View, Calif., US), including quantitative real time reverse transcriptase PCR (e.g. qRT-PCR, See Woudenberg T, et al., U.S. Pat. No. 5,928,907 and U.S. Pat. No. 6,015,674),
3) single-molecule detection (See Neely L, et al., Nat. Methods. 2006; 3(1):41-46, Chan E, U.S. Pat. No. 6,355,420, Chandler M, et al., U.S. Pat. No. 6,916,661, and Chandler D, et al., U.S. Pat. No. 6,632,526),
4) bead-based flow cytometric methods (See Lu J, et al., Nature 2005; 435(7043):834-838 and Chandler V, et al., U.S. Pat. No. 6,524,793), and
5) assays using nucleic acids. See Nelson P, et al., Nat. Methods 2004; 1(2):155-161, Wu Y, et al., RNA 2007; 13(1):151-159, Lader E, et al., U.S. Pat. No. 6,057,134, Brown D, et al., U.S. Pat. No. 6,891,032, and Delenstarr; G, et al., U.S. Pat. No. 7,122,303.

Total cellular RNA can be purified from cells by homogenization in the presence of a nucleic acid extraction buffer, followed by centrifugation. Nucleic acids are precipitated, and DNA is removed by treatment with DNase and precipitation. Nucleic acids, especially RNA and specifically miRNA, can be isolated by using any techniques known in the art. There are two main methods for isolating RNA: i) phenol-based extraction and ii) silica matrix or glass fiber filter (GFF)-based binding. Phenol-based reagents contain a combination of denaturants and RNase inhibitors for cell and tissue disruption and subsequent separation of RNA from contaminants. Phenol-based isolation procedures can recover RNA species in the 10-200-nucleotide range (e.g. miRNAs, 5S rRNA, 5.8S rRNA, and U1 snRNA). If a sample of “total” RNA was purified by the popular silica matrix column or GFF procedure, it may be depleted in small RNAs. Extraction procedures such as those using Trizol or TriReagent, however will purify all RNAs, large and small, and are the recommended methods for isolating total RNA from biological samples that will contain miRNAs/siRNAs. Any method required for the processing of a sample prior to quantifying the level of the miRNAs according to the method of the invention falls within the scope of the present invention. These methods are known in the art.

RNA molecules can be separated by gel electrophoresis on agarose gels according to standard techniques, and transferred to nitrocellulose filters (e.g. Northern blot). The RNA is then immobilized on the filters by heating. Detection and quantification of specific RNA is accomplished using appropriately labeled DNA or RNA probes complementary to the RNA in question. See Sambrook J, et al., Molecular Cloning. A Laboratory Manual (Cold Spring Harbor Laboratory Press, Cold Spring Harbor, N.Y., US, 1989),

Suitable probes for Northern blot hybridization of a given miRNA gene product can be produced using the nucleotide sequence of a miRNA. miRNA, hairpin pre-miRNA and miRNA* sequences known in the art from the miRBase::Sequences database.

Methods for preparation of labeled DNA and RNA probes, and the conditions for hybridization thereof to target nucleotide sequences are known in the art. See Sambrook, 1989, supra. For example, the nucleic acid probe can be labeled with a radionuclide (e.g. 3H, 32P, 33P, 14C, or 35S), a heavy metal, a ligand capable of functioning as a specific binding pair member for a labeled ligand (e.g. biotin, avidin or an antibody), a fluorescent molecule, a chemiluminescent molecule, or an enzyme.

Probes can be labeled to high specific activity by either the nick translation method or the random priming method. See Rigby P, et al., J. Mol. Biol. 1977; 113:237-251 and Fienberg A, et al., Anal. Biochem. 1983; 132:6-13. The latter is the method of choice for synthesizing 32P-labeled probes from single-stranded DNA or RNA templates. For example, by replacing pre-existing nucleotides with highly radioactive nucleotides according to the nick translation method, it is possible to prepare P-labeled nucleic acid probes with a specific activity well in excess of 10 cpm/ng. Autoradiographic detection of hybridization can be performed then by exposing hybridized filters to photographic film. Densitometric scanning of the photographic films exposed by the hybridized filters provides an accurate measurement of miRNA gene transcript levels. Using another approach, miRNA gene transcript levels can be quantified by computerized imaging systems, such as a Molecular Dynamics 400-B 2D PhosphorImager (Amersham Biosciences, Inc., Piscataway, N.J., US).

When radionuclide labeling of DNA or RNA probes is not practical, the random-primer method can be used to incorporate an analog such as, for example, the dTTP analog 5-(N—(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g. anti-biotin antibodies) coupled to fluorescent dyes or enzymes that produce color reactions. In addition to Northern and other RNA blotting hybridization techniques, determining the levels of RNA transcripts can be accomplished using the technique of in situ hybridization. This technique requires fewer cells than the Northern blotting technique, and involves depositing whole cells onto a microscope cover slip and probing the nucleic acid content of the cell with a solution containing radioactive or otherwise labeled nucleic acid (e.g. cDNA or RNA) probes. This technique is particularly well-suited for analyzing tissue biopsy samples. See Gewirtz A, et al., U.S. Pat. No. 5,427,916. Suitable probes for in situ hybridization of a given miRNA gene product can be produced by using the nucleotide sequence of a miRNA. miRNA, miRNA* and pre-miRNA hairpin sequences known in the art correspond to the miRNAs, miRNA*s and pre-miRNA hairpins described in the miRBase::Sequences.

The relative number of miRNA gene transcripts in cells can also be determined by reverse transcription of miRNA gene transcripts, followed by amplification of the reverse-transcribed transcripts by polymerase chain reaction (RT-PCR).

The levels of miRNA gene transcripts can be quantified by comparing them to an internal standard such as, for example, the level of mRNA from a “housekeeping” gene present in the same sample. A suitable “housekeeping” gene for use as an internal standard includes, without limitation, myosin or glyceraldehyde-3-phosphate dehydrogenase (GAPDH). The methods for performing quantitative RT-PCR and variations thereof are known in the art.

The three most commonly used methods of quantitative polymerase chain reaction are: i) an agarose gel electrophoresis, ii) the use of SYBR Green (a double stranded DNA dye), and iii) the use of a fluorescent reporter probe. The latter two can be performed in real-time.

The SYBR Green dye method is based on a DNA binding dye that attaches to newly synthesized double stranded (ds)DNA. The dye only fluoresces when bound to dsDNA. Then, by making reference to a standard sample or a standard curve, the dsDNA concentration in the sample can be determined. A drawback of this technique is that SYBR Green will label all dsDNA including any unexpected PCR products and primer dimers, leading to potential complications and wrong measurements.

The fluorescent reporter probe method is the most accurate and reliable qPCR technique. It is based on the use of a sequence-specific nucleic acid probe that quantifies its complementary sequence only and not all double stranded DNA. It is commonly carried out with DNA based probes with a fluorescent reporter and a quencher held in adjacent positions, so-called dual-labeled probes. The close proximity of the reporter to the quencher prevents its fluorescence; it is only on the breakdown of the probe that the fluorescence is detected. This process depends on the 5′ to 3′ exonuclease activity of the polymerase involved. The real-time quantitative PCR reaction is prepared with the addition of the dual-labeled probe. On denaturation of the double-stranded DNA template, the probe is able to bind to its complementary sequence in the region of interest of the template DNA (as the primers will, too). When the PCR reaction mixture is heated to activate the polymerase, the polymerase starts synthesizing the complementary strand to the primed single stranded template DNA. As the polymerization proceeds it reaches the probe bound to its complementary sequence, which is then hydrolyzed by the action of the 5′-3′ exonuclease activity of the polymerase, thus separating the fluorescent reporter and the quencher molecules. This results in a detectable increase in fluorescence, which makes possible to determine accurately the initial and final DNA quantities.

Any PCR method useful for determining the expression of a nucleic acid molecule as defined herein falls within the scope of the present invention. In a preferred embodiment, a real-time quantitative RT-PCR method is used for the detection and quantification of the nucleic acids of the invention. More preferably, the RT-PCR method is based on the use of the SYBR Green dye or a dual-labeled probe.

In some embodiments, determining simultaneously the expression level of a plurality of different of miRNAs may be convenient. In other instances, it may be desirable to determine the expression level of miRNAs or their transcripts in a plurality of samples. Assessing expression levels for hundreds of miRNAs is time consuming and requires a large amount of total RNA (at least 20 ng for each Northern blot) and autoradiographic techniques that require radioactive isotopes. To overcome these limitations, an oligolibrary in microchip format may be constructed containing a set of probe oligonucleotides specific for a set of miRNA genes

A set of mature miRNAs, miRNA*s, and pre-miRNA hairpin precursors known in the art may be found in Tables 1-3. The nucleic acid sequences corresponding to the miRNAs, hairpin pre-miRNAs and miRNA*s described in Tables 1-3 are available from the miRBase:Sequences database of the Wellcome Trust Sanger Institute (http://www.mirbase.org/index.shtml, July 2012). The microchip is prepared from gene-specific oligonucleotide probes generated from known miRNAs. According to one embodiment, the array contains two different oligonucleotide probes for each miRNA, one containing the active sequence and the other being specific for the precursor of the miRNA. The array may also contain controls such as one or more (e.g. mouse) sequences differing from (e.g. human) orthologs by only a few bases, which can serve as controls for hybridization stringency conditions. tRNAs from both species may also be printed on the microchip, providing an internal, relatively stable positive control for specific hybridization. One or more appropriate controls for non-specific hybridization may also be included on the microchip. For this purpose, sequences are selected based upon the absence of any homology with any known miRNAs.

The microchip may be fabricated by techniques known in the art. For example, probe oligonucleotides of an appropriate length (e.g. 40 nucleotides) may be 5′-amine modified at position C6 and printed using commercially available microarray systems (e.g. GeneMachine OmniGrid™ 100 Microarrayer, Amersham CodeLink™ activated slides). Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions (e.g. 6×SSPE/30% formamide at 25° C. for 18 hours, followed by washing in 0.75×TNT at 37° C. for 40 minutes). Hybridization occurs at positions on the array where the immobilized probe DNA recognizes a complementary target cDNA in the sample. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary miRNAs, in the sample. According to one embodiment, the labeled cDNA oligomer is a biotin-labeled cDNA, prepared from a biotin-labeled primer. The microarray is then processed by direct detection of the biotin-containing transcripts by using, for example, a Streptavidin-Alexa647 conjugate, and scanned utilizing conventional scanning methods. The intensity of each spot on the array is proportional to the abundance of the corresponding miRNA in the sample.

The use of the array has several advantages for miRNA expression detection. First, the global expression of several hundred genes can be identified in a single sample at one time point. Second, through careful design of the oligonucleotide probes, the expression of both mature and precursor molecules can be identified. Third, in comparison with Northern blot analysis, the chip requires a small amount of RNA, and provides reproducible results using 2.5 μg of total RNA. The relatively limited number of miRNAs (a few hundred per species) potentially allows the construction of a common microarray for several species, with distinct oligonucleotide probes for each. Such a tool would allow for analysis of trans-species expression for each known miRNA under various conditions. The microarray methods described herein are useful for both the identification of miRNA signatures and the diagnostic and therapeutic applications of the invention.

In the method of the invention, the assaying of the cells or biological samples described above can further comprise the performance of suitable controls known in the art. The relative miRNA expression in the control or normal samples can further be determined with respect to one or more RNA expression standards. The standards can comprise, for example, a zero miRNA gene expression level, the miRNA gene expression level in a standard cell line, or the average level of miRNA gene expression previously obtained for a population of normal human controls. In a particular embodiment, the sample is a body fluid such as a blood. Preferably, the sample comprises peripheral blood mononuclear cells (PBMC) from the subject.

In a second step, the method for the identification of HIV controller subjects comprises comparing the expression levels of one or more of the above miRNAs with reference values for each miRNA, wherein increased expression level of said miRNA with respect to a reference value is indicative that the patient is a HIV controller. In a preferred embodiment, the reference sample is obtained from a viremic progressor or from a HIV-1 infected subject undergoing ART.

Chronic progressor HIV infected individuals exhibit a high viral load, for example, plasma HIV RNA levels>10,000 copies/mL, as compared to an individual not infected with HIV. The viral load of a chronic progressor increases over time, for example, over several months (e.g. 1, 2, 3, 4, 5, 6, 7, 8, 9, 10, 11 or 12 months) or years (e.g. 2, 3, 4, 5, 6, 7, 8, 9, or 10 years).

Alternatively, the reference sample may be obtained by pooling samples from viremic progressors or by pooling samples from subjects undergoing ART. The expression profile of the miRNAs in the reference sample can be generated; preferably, from a population of two or more subjects (e.g. the population can comprise 3, 4, 5, 10, 15, 20, 30, 40, 50 or more subjects).

2. Therapeutic Uses of the miRNAs

In yet another aspect, the invention relates to a miRNA selected from the group consisting of miR-27a, miR-27b, miR-29b, miR-125, miR-146, miR-155 and miR-221 or a polynucleotide encoding said miRNA for use in the treatment or prevention of a disease caused by HIV. Alternatively, the invention relates to the use of a miRNA selected from the group consisting of miR-27a, miR-27b, miR-29b, miR-125, miR-146, miR-155 and miR-221 or a polynucleotide encoding said miRNA for the manufacture of a medicament for the treatment of a disease caused by HIV. Alternatively, the invention relates to a method for the treatment of a disease caused by HIV in a subject in need thereof comprising the administration to said subject a composition comprising a miRNAs selected from the group consisting of miR-27a, miR-27b, miR-29b, miR-125, miR-146, miR-155 and miR-221 or a polynucleotide encoding said miRNA. In some embodiments, the polynucleotide comprising one or more miRNA sequences is conjugated to a steroid, such as cholesterol, a vitamin, a fatty acid, a carbohydrate or glycoside, a peptide, or another small molecule ligand. In certain embodiments, an agonist of one or more miRNAs is an agent distinct from the miRNA itself that acts to increase, supplement, or replace the function of the one or more miRNAs. The miRNA or the polynucleotide according to the invention may further comprise a pharmaceutically acceptable carrier.

In some embodiments, the DNA molecule encoding the miRNA or precursor thereof is found in an expression cassette. The expression cassette may comprise one or more regulatory sequences, selected on the basis of the cells to be used for expression, operably linked to a polynucleotide encoding the miRNA or precursor thereof.

The appropriate promoter or regulatory sequence can be readily selected to allow the expression of the relevant miRNA or precursor thereof in the cell of interest. In certain embodiments, the promoter utilized to direct intracellular expression of a miRNA or precursor thereof is a RNA polymerase III (Pol III) promoter. See Yu J, et al., Proc. Natl. Acad. 2002; 99(9):6047-6052, Sui Y, et al., Proc. Natl. Acad. Sci. 2002; 99(8):5515-5520, Paddison P, et al., Genes Dev. 2002; 16:948-958, Brummelkamp T, et al., Science 2002; 296:550-553, Miyagashi M, et al., Biotech. 2002; 20:497-500, Paul C, et al., Nat. Biotech. 2002; 20:505-508, and Tuschl T, et al., Nat. Biotech. 2002; 20:446-448. In another embodiment, a RNA polymerase I promoter (e.g. a tRNA promoter) can be used, instead. See McCown M, et al., Virology 2003; 313(2):514-524 and Kawasaki H, et al., Nucleic Acids Res. 2003; 31(2):700-707. The regulatory functions can also be provided by viral regulatory elements.

For example, commonly used promoters are derived from polyoma, adenovirus 2, cytomegalovirus, and simian virus 40. Other suitable expression systems for both prokaryotic and eukaryotic cells are described in the art. See Sambrook, 1989, and Goeddel, 1990, supra. In vitro transcription can be performed using a variety of systems commercially available (e.g. T7, SP6, and T3 promoter/polymerase systems from Promega Corp. (Madison, Wis., US), Clontech Labs, Inc. (Mountain View, Calif., US), and New England Biolabs Inc. (Ipswich, Mass., US)). Vectors including the T7, SP6, or T3 promoter are well known in the art and can readily be modified to direct transcription of miRNAs or precursors thereof. When double-stranded miRNA precursors are synthesized in vitro, the strands can be allowed to hybridize before introducing into a cell or before administration to a subject. As noted above, miRNAs or precursors thereof can be delivered or introduced into a cell as a single RNA molecule including self-complementary portions (e.g. an shRNA that can be processed intracellularly to yield a miRNA), or as two strands hybridized to one another. In other embodiments, the miRNAs or precursors thereof are transcribed in vivo. Regardless of the mechanism of translation, a primary transcript may be obtained from miRNA or a precursor thereof either in vivo or in vitro. The transcript can then be processed (e.g. by one or more cellular enzymes) to inhibit miRNA.

Amounts effective for therapeutic use can depend on the severity of the disease and the age, weight, general state of the patient, and other clinical factors. Thus, the final determination of the appropriate treatment regimen will be made by the attending clinician. Typically, dosages used in vitro can provide useful guidance in the amounts useful for in situ administration of the pharmaceutical composition, and animal models may be used to determine effective dosages for treatment of particular disorders. See Gilman R, et al., Eds., Goodman and Gilman's: The Pharmacological Basis of Therapeutics, 8th Ed. (Pergamon Press, Inc., New York, N.Y., US, 1990), and Gennaro A, Ed., Remington's Pharmaceutical Sciences, 18th Ed. (Mack Publishing Co., Easton, Pa., US, 1990). Typically, the dose range for a miRNA is from about 0.1 ng/kg body weight to about 100 mg/kg body weight. Other suitable ranges include doses of from about 1 ng/kg to 10 mg/kg body weight. In one example, the dose is about 1.0 ng to about 50 mg, for example, 1 ng to 1 mg, such as 1 mg miRNA per subject. The dosing schedule can vary from daily to as seldom as once a year, depending on clinical factors, such as the subject's sensitivity to the peptide and tempo of their disease. Therefore, a subject can receive a first dose of a disclosed therapeutic molecule, and then receive a second dose (or even more doses) at some later time(s), such as at least one day later, such as at least one week later.

All forms of HIV-1 are potentially treatable with the compounds of the present invention. The compounds of the present invention are useful for treating protease inhibitor resistant HIV, reverse transcriptase inhibitor resistant HIV, and entry or fusion inhibitor resistant HIV. These compounds are also useful in the treatment of HIV groups M, N, and O; HIV-1 subtypes, including the A1, A2, B, C, D, F1, F2, G, H, and J subtypes; and circulating recombinant HIV forms. The compounds of the present invention are useful for treating CCR5 tropic HIV strains, as well as CXCR4 tropic HIV strains.

In another embodiment, the present invention refers to a pharmaceutical composition comprising at least one miRNA of the invention, a polynucleotide encoding at least one miRNA or a precursor thereof and a pharmaceutically acceptable carrier.

The pharmaceutical compositions disclosed herein can be prepared and administered in dose units. Solid dose units include tablets, capsules, transdermal delivery systems, and suppositories. The administration of a therapeutic amount can be carried out both by single administration in the form of an individual dose unit or else several smaller dose units and also by multiple administrations of subdivided doses at specific intervals. Suitable single or divided doses include, but are not limited to about 0.01, 0.1, 0.5, 1, 3, 5, 10, 15, 30, or 50 μg pharmaceutical composition/kg/day.

The nucleic acid constructs encoding antigenic the miRNAs described herein are used, for example, in combination, as pharmaceutical compositions (medicaments) for use in therapeutic, for example, prophylactic regimens (e.g. vaccines) and administered to subjects (e.g. primate subjects, such as human subjects) to elicit an immune response against one or more Glade or strain of HIV. For example, the compositions described herein can be administered to a human (or non-human) subject prior to infection with HIV to inhibit infection by or replication of the virus. Thus, the pharmaceutical compositions described above can be administered to a subject to elicit a protective immune response against HIV. To elicit an immune response, a therapeutically effective (e g immunologically effective) amount of the nucleic acid constructs are administered to a subject, such as a human (or non-human) subject.

The nucleic acid molecules encoding the miRNAs of the invention can be delivered intracellularly. For instance, the nucleic acid may be administered by means of a retroviral vector, direct injection, microparticle bombardment (e.g. a gene gun), coating with lipids, cell-surface receptors or transfecting agents, or by administering it in linkage to a homebox-like peptide in such a way as to favor cellular penetration. See Morgan J, et al., U.S. Pat. No. 4,980,286, and Joliot A, et al., Proc. Natl. Acad. Sci. USA 1991; 88:1864-1868. The present invention includes all forms of nucleic acid delivery, including synthetic oligos, naked DNA, plasmid and viral vector, episomal or chromosomally integrated.

In another approach to using nucleic acids, the miRNAs can be expressed by attenuated viral hosts or vectors or bacterial vectors. Recombinant vaccinia virus, adeno-associated virus (AAV), herpes virus, retrovirus, or other viral vectors can be used to express the peptide or protein, thereby eliciting a CTL response.

In one example, a viral vector is utilized. These vectors include, but are not limited to, adenovirus, herpes virus, vaccinia, or an RNA virus such as a retrovirus. In one example, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). When the subject is a human, a vector such as the gibbon ape leukemia virus (GaLV) can be utilized. A number of additional retroviral vectors can incorporate multiple genes. All of these vectors can transfer or incorporate a gene for a selectable marker so that transduced cells can be identified and generated. By inserting a nucleic acid sequence encoding a miRNA into the viral vector, along with another gene that encodes the ligand for a receptor on a specific target cell, for example, the vector may become target specific. Retroviral vectors can be made target specific by attaching, for example, a sugar, a glycolipid, or a protein. Preferred targeting is accomplished by using an antibody to target the retroviral vector. The specific polynucleotide sequences which can be inserted into the retroviral genome or attached to a viral envelope to allow the delivery of the polynucleotide encoding a miRNA can be readily ascertained without undue experimentation.

Suitable formulations for nucleic acid constructs, include aqueous and non-aqueous solutions, isotonic sterile solutions, which can contain anti-oxidants, buffers, and bacteriostats, and aqueous and non-aqueous sterile suspensions that can incorporate suspending agents, solubilizers, thickening agents, stabilizers, and preservatives. The formulations can be presented in unit-dose or multi-dose sealed containers, such as ampoules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. More preferably, the composition of the invention is formulated to protect the nucleic acid constructs from damage prior to administration. For example, the composition can be formulated to reduce loss of the adenoviral vectors on devices used to prepare, store, or administer the expression vector, such as glassware, syringes, or needles. The compositions can be formulated to decrease the light sensitivity or temperature sensitivity of the components. Preferably, the composition comprises a pharmaceutically acceptable liquid carrier such as, for example, those described above, and a stabilizing agent selected from the group consisting of polysorbate 80, L-arginine, polyvinylpyrrolidone, trehalose, and combinations thereof.

In therapeutic applications, a therapeutically effective amount of a composition of the invention is administered to a subject prior to or following exposure to or infection by HIV. When administered prior to exposure, the therapeutic application can be referred to as a prophylactic administration (e.g. vaccine). Single or multiple administrations of the compositions are administered depending on the dosage and frequency as required and tolerated by the subject. In one embodiment, the dosage is administered once as a bolus. In another embodiment, the dosage can be applied periodically until a therapeutic result, such as a protective immune response, is achieved. Generally, the dose is sufficient to treat or ameliorate symptoms or signs of disease without producing unacceptable toxicity to the subject. Systemic or local administration can be utilized.

Nucleic acid constructs encoding the miRNAs for use in the present invention can be introduced in vivo as naked DNA plasmids. DNA vectors can be introduced into the desired host cells by methods known in the art, including but not limited to transfection, electroporation (e.g. transcutaneous electroporation), microinjection, transduction, cell fusion, DEAE dextran, calcium phosphate precipitation, use of a gene gun, or use of a DNA vector transporter. See Wu C, et al., J. Biol. Chem. 1992; 267:963-967, Wu C and Wu G, Biol. Chem. 1988; 263:14621-14624, and Williams R, et al., Proc. Natl. Acad. Sci. USA 1991; 88:2726-2730. Methods for formulating and administering naked DNA to mammalian muscle tissue have been described also. See Felgner P, et al., U.S. Pat. No. 5,580,859, and U.S. Pat. No. 5,589,466. There are other molecules useful for transfecting cells with nucleic acids in vivo, such as cationic oligopeptides, peptides derived from DNA binding proteins, or cationic polymers. See Bazile D, et al., WO1995021931, and Byk G, et al., WO1996025508.

Another known method for introducing nucleic acid constructs encoding miRNAs into host cells is particle bombardment (aka biolistic transformation). Biolistic transformation is commonly accomplished in one of several ways. One common method involves propelling inert or biologically active particles at cells. See Sanford J, et al., U.S. Pat. No. 4,945,050, U.S. Pat. No. 5,036,006, and U.S. Pat. No. 5,100,792.

Alternatively, the vector can be introduced in vivo by lipofection. The use of cationic lipids can promote encapsulation of negatively charged nucleic acids, and also promote fusion with negatively charged cell membranes. See Felgner P, Ringold G, Science 1989; 337:387-388. Particularly useful lipid compounds and compositions for transfer of nucleic acids have been described previously. See Felgner P, et al., U.S. Pat. No. 5,459,127, Behr J, et al., WO1995018863, and Byk G, WO1996017823.

The nucleic acid encoding the miRNA of the invention may be administered in a single dose, or multiple doses separated by a time interval in a manner similar to the administration of the miRNA. For example, two doses, or three doses, or four doses, or five doses, or six doses or more can be administered to a subject over a period of several weeks, several months or even several years, to optimize the immune response.

The present invention also contemplates the administration of the compositions of the invention in combination with other suitable therapeutic treatments which are useful for treating a disease caused by HIV infection. Thus, the invention also relates to a composition or kit-of-parts comprising a composition and one or more drugs used for the treatment of a disease caused by HIV infection. Preferable, the composition is immunogenic.

The pharmaceutical compositions of the invention may be applied to the vagina in a number of forms including, but not limited to, aerosols, foams, sprays, pastes, gels, jellies, creams, suppositories, tablets, pessaries, tampons, and devices such as vaginal rings. They can be in the form of immediate release or controlled release formulations. Preferably, creams and gels are utilized. See Vickery B, et al., U.S. Pat. No. 4,368,186, Gazzani G, U.S. Pat. No. 4,371,518, Tice T, et al., U.S. Pat. No. 4,389,330, Joyce C, et al., U.S. Pat. No. 4,415,585, and Riley T, et al., U.S. Pat. No. 4,551,148.

In a particularly preferred embodiment, the pharmaceutical composition is applied topically to the vagina. Typically, the topical application is carried out prior to the beginning of vaginal intercourse, suitably 0 to 60 minutes, preferably 0 to 5 minutes, prior to the beginning of vaginal intercourse. The application may be carried out into and around the vagina and vaginal area (e.g. the individual anatomical parts, such as, labia majora, labia minora, clitoris) of a female. In a preferred embodiment, the pharmaceutical composition is vaginal cream.

Pharmaceutical creams, as known in the art, are viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant.

A preferred class of bioadhesive gelling polymers to be used according to this invention is that comprised of acrylic acid polymers crosslinked with allyl sucrose or allyl ethers of pentaerythritol (e.g. Carbopol, Goodrich Corp. Akron, Ohio, US). These bioadhesive gelling polymers are commercially available. Carbopol 934P and 97 IP are usually preferred for vaginal administration. Another preferred class of bioadhesive gelling polymer to be used in the present invention is that comprised of acrylic acid polymers crosslinked with divinyl glycol, such as Noveon® AA-1 Polycarbophil, USP (The Lubrizol Corp., Wickliffe, Ohio, US). Many of this type of bioadhesive gelling polymers are commercially available.

For example, suitable vehicle bases include, but are not limited to, hydrocarbon bases or oleaginous bases, absorption bases, water-removable bases and water-soluble bases. In some embodiments, the vehicle base is non-irritating, non-staining, stable, non-pH dependent or compatible with the compounds of the invention.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by the admixture of the active ingredient with the softened or melted carrier(s) followed by chilling and shaping in moulds.

In another embodiment, the present invention involves topical administration of a composition of the invention to the anus. The composition administered to the anus is suitably a foam, cream, or jelly such as those described above with regard to vaginal application. In the case of anal application, it may be preferred to use an applicator which distributes the composition substantially evenly throughout the anus. For example, a suitable applicator is a tube 2.5 to 25 cm, preferably 5 to 10 cm, in length having holes distributed regularly along its length.

In another embodiment, the present method may be carried out by applying the composition of the invention orally. Oral application is suitably carried out by applying a composition which is in the form of a mouthwash or gargle. Oral application is especially preferred to prevent infection during dental procedures. Suitably, the composition is applied just prior to the beginning of the dental procedure and periodically throughout the procedure. Formulations suitable for topical administration in the mouth include lozenges comprising the active ingredient in a flavored base, usually sucrose and acacia or tragacanth; pastilles comprising the active ingredient in an inert basis such as gelatin and glycerin, or sucrose and acacia; and mouthwashes comprising the active ingredient in a suitable liquid carrier.

In the case of a mouthwash or gargle, it may be preferred to include in the composition an agent which will mask the taste or odor of the compound of the invention. Such agents include those flavoring agents typically found in mouthwashes and gargles, such as spearmint oil, or cinnamon oil.

The therapeutically effective amount of the compound or compounds of the invention to be administered will generally depend, among other factors, on the chosen method of administration. For this reason, the doses mentioned in this invention must only be considered as guidelines for the person skilled in the art. It is understood that animal studies may be performed to determine an appropriate dosage amount.

It is noted that when the composition is in the form of a suppository (including vaginal suppositories), the suppository will usually be 1 to 5 g, preferably about 3 g, and the entire suppository will be applied. A vaginal tablet will suitably be 1 to 5 g, preferably about 2 g, and the entire tablet will be applied. When the composition is vaginal cream, suitably 0.1 to 2 g, preferably about 0.5 g of the cream will be applied. When the composition is a water-soluble vaginal cream, suitably 0.1 to 2 g, preferably about 0.6 g, are applied. When the composition is a vaginal spray-foam, suitably 0.1 to 2 g, preferably about 0.5 g, of the spray-foam are applied. When the composition is an anal cream, suitably 0.1 to 2 g, preferably about 0.5 g of the cream are applied. When the composition is an anal spray-foam, suitably 0.1 to 2 g, preferably about 0.5 g of the spray-foam are applied. When the composition is a mouthwash or gargle, suitably 1 to 10 mL, preferably about 5 mL are applied.

In a preferred embodiment of the invention, the pharmaceutical composition comprises from about 0.1 μg to about 300 mg of a compound of the invention. More preferably, the composition comprises from about 1 μg to about 30 mg of a compound of the invention.

The present compositions may also be in the form of a time-release composition. In this embodiment, the compound of the invention is incorporated in a composition which will release the active ingredient at a rate which will result in an effective vaginal or anal concentration of said compound. See in Lew D, Eds., Controlled Release of Pesticides and Pharmaceuticals (Plenum Press, New York, N.Y., US, 1981), Bodmeier R, et al., J. Pharm. Sci. 1989; 78(1):964-967, Inc., Amies C, et al., J. Pathol. Bacteriol. 1959; 77(2):435-442, Pfister G, et al., J. Controlled Release 1986, 3:229-233, Behan J, et al., U.S. Pat. No. 5,185,155, Viegas T, et al., U.S. Pat. No. 5,143,731, and Lance W, U.S. Pat. No. 5,248,700.

The present compositions may also be in a form which releases the compounds of the invention in response to some event such as vaginal or anal intercourse. For example, the composition may contain the compounds of the invention in vesicles or liposomes, which are disrupted by the mechanical action of intercourse. Compositions comprising liposomes have been described previously. See Janoff A, et al., U.S. Pat. No. 5,231,112 and Deamer D, et al., “Liposome Preparation: Methods and Mechanisms”, in Liposomes, (Marcel Dekker Inc., New York, N.Y., US, 1983, pp. 27-51); Sessa J, et al., J. Biol. Chem. 1970; 245: 3295-3300; Sessa J, et al., J. Pharmaceutics Pharmacol. 1982; 34:473-474; and Breimer D, Speiser P, Eds., Topics in Pharmaceutical Sciences (Elsevier B. V., New York, N.Y., US, 1985, pp. 345-358).

The present compositions may be also delivered via an article, such as an intrauterine device (IUD), vaginal diaphragm, vaginal ring, vaginal sponge, pessary, or condom. In the case of an IUD or diaphragm, a time-release or mechanical-release composition may be preferred, while in the case of condoms, a mechanical-release composition is preferred.

In another embodiment, the present invention provides novel articles, which are useful for the prevention of HIV infection. In particular, the present articles are those which release the compounds of the invention when placed on an appropriate body part or in an appropriate body cavity. Thus, the present invention provides IUDs, vaginal diaphragms, vaginal sponges, pessaries, or condoms which contain or are associated with a compound of the invention.

Thus, the present article may be an IUD containing one or more compounds of the invention. Suitable IUDs are disclosed in art. See Ramwell P, U.S. Pat. No. 3,888,975 and Berthet J, et al., U.S. Pat. No. 4,283,325. The present article may be an intravaginal sponge which comprises and releases, in a time-controlled fashion, a compound of the invention. See Robinson T, U.S. Pat. No. 3,916,898 and Barrows T, U.S. Pat. No. 4,360,013. The present article may also be a vaginal dispenser, which releases the compound of the invention. See Wong P, U.S. Pat. No. 4,961,931.

The present article may also be a condom coated with a compound of the invention. In a preferred embodiment, the condom is coated with a lubricant or penetration enhancing agent which comprises a compound the invention and a spermicide, which is optionally selected from benzalkonium chloride, benzethonium chloride, cetyl pyridinium chloride, methylbenzethonium chloride, tetra-decyltrimethyl ammonium bromide, benzalkonium bromide, monylphenyl ethers, lauryl ethers, and octoxynols. However, it is recommended that use of a condom should be associated with use of an appropriate lubricating agent (i.e. one that does not degrade the mechanical strength properties of the condom and that does not increase its porosity due to the latex being attacked). See Marquardt G, EP 0457127, Aguadisch L, et al., EP 0475664, Copper E, U.S. Pat. No. 4,537,776, Copper E, et al., U.S. Pat. No. 4,552,872, Copper E, et al., U.S. Pat. No. 4,954,487, and Kelly P, U.S. Pat. No. 5,208,031.

3. Microbicide Compositions of the Invention

The miRNAs forming part of the profile according to the present invention are capable of discriminating HIV controller patients from patients which suffer viral progression. Thus, the invention also contemplates the use of the miRNAs for increasing the antiviral activity of anti-HIV agents. Thus, in another aspect, the invention relates to a microbicide composition or kit-of-parts comprising at least one miRNAs selected from the group consisting of miR-27a, miR-27b, miR-29b and miR-221 or a polynucleotide encoding said miRNA or a polynucleotide encoding said miRNA. Preferably, the composition or kit-of-parts comprises also an anti-HIV agent.

Suitable anti-HIV agents for use according to the present invention include, without limitation, HIV protease inhibitors, HIV reverse transcriptase inhibitors, HIV entry inhibitors and HIV immunogens.

Preferred protease inhibitors for use in combination with a compound of the present invention include saquinavir, ritonavir, indinavir, nelfhavir, amprenavir, lopinavir, atazanavir, darunavir, brecanavir, fosamprenavir, and tipranavir. Particularly useful such combinations include, for example, AZT+3TC; TDF+3TC; TDF+FTC; ABC+3TC; and abacavir+3TC.

Suitable reverse transcriptase inhibitors for use in the compositions according to the present invention is one or more compounds selected from the group consisting of emtricitabine, capravirine, tenofovir, lamivudine, zalcitabine, delavirdine, nevirapine, didanosine, stavudine, abacavir, alovudine, zidovudine, racemic emtricitabine, apricitabine, emivirine, elvucitabine, TMC-278, DPC-083, amdoxovir, (−)-beta-D-2,6-diamino-purine dioxolane, MIV-210 (FLG), DFC (dexelvucitabine), dioxolane thymidine, Calanolide A, etravirine (TMC-125), L697639, atevirdine (U87201E), MIV-150, GSK-695634, GSK-678248, TMC-278, KP1461, KP-1212, lodenosine (FddA), 5-[(3,5-dichlorophenyl)thio]-4-isopropyl-1-(4-pyridylmethyl)imidazole-2-methanol carbamic acid, (−)-I²-D-2,6-diaminopurine dioxolane, AVX-754, BCH-13520, BMS-56190 ((4S)-6-chloro-4-[(1E)-cyclopropylethenyl]-3,-4-dihydro-4-trifluoromethyl-2(1H)-quinazolinone), TMC-120, and L697639, where the compounds are present in amounts effective for treatment of HIV when used in a combination therapy.

In some embodiments of the present invention, the viral entry inhibitor is a fusion inhibitor, a CD4 receptor binding inhibitor, is a CD4 mimic or a gp120 mimic In some further embodiments, the viral entry inhibitor is a gp41 antagonist, a CD4 monoclonal antibody or a CCR5 antagonist, including CCR5 antagonist sub-classes such as, for example, zinc finger inhibitors. In yet another embodiment, the viral entry inhibitor is a CXCR4 co-receptor antagonist.

Suitable HIV immunogens for use according to the present invention include HIV envelope (env; e.g. NCBI Ref. Seq. NPJ357856), gag (e.g. p6, p7, p17, p24, GenBank AAD39400J), the protease encoded by pol (e.g. UniProt P03366), nef (e.g. fenBank-CAA41585J; Shugars D, et al., J. Virol. 1993; 67(8):4639-4650), as well as variants, derivatives, and fusion proteins thereof as described previously. See Gómez J, et al., Vaccine 2007; 25(29): 69-71. Immunogens (e.g. env and pol) may be combined as desired. Immunogens from different HIV isolates. Suitable strains and combinations may be selected by a person skilled in the art as desired.

An immune response to HIV may be measured by several means known in the art such as, for example, viral load, T-cell proliferation, T-cell survival, cytokine secretion by T-cells, or an increase in the production of antigen-specific antibodies.

Additionally, the compositions according to the present invention may further comprise an antiretroviral agent selected from the group consisting of vaccines, gene therapy treatments, cytokines, TAT inhibitors, and immunomodulators in amounts effective for treatment of HIV when used in a combination therapy.

Additionally, the compositions according to the present invention may further comprise an antiinfective agent selected from the group consisting of antifungals, antibacterials, anti-neoplastics, anti-protozoals, DNA polymerase inhibitors, DNA synthesis inhibitors, anti-HIV antibodies, HIV antisense drugs, IL-2 agonists, α-glucosidase inhibitors, purine nucleoside phosphorylase inhibitors, apoptosis agonists, apoptosis inhibitors, and cholinesterase inhibitors, where the compounds are present in amounts effective for treatment of HIV when used in a combination therapy.

Additionally, the compositions according to the present invention may further comprise an immunomodulator selected from the group consisting of pentamidine isethionate, autologous CD8+ infusion, γ-interferon immunoglobulins, thymic peptides, IGF-I, anti-Leu3A, auto vaccination, biostimulation, extracorporeal photophoresis, cyclosporin, rapamycin, FK-565, FK-506, GCSF, GM-CSF, hyperthermia, isopinosine, rVIG, HIVIG, passive immunotherapy and polio vaccine hyperimmunization, where the compounds are present in amounts effective for treatment of HIV when used in a combination therapy.

The microbicide compositions are suitable for preventing or reducing symptoms associated with HIV infection. These include symptoms associated with the minor symptomatic phase of HIV infection, including, for instance, shingles, skin rash and nail infection, mouth sores, recurrent nose and throat infection, and weight loss. In addition, further symptoms associated with the major symptomatic phase of HIV infection, include, for example, oral and vaginal thrush (e.g. candidiasis), persistent diarrhea, weight loss, persistent cough, reactivated tuberculosis, and recurrent herpes infections, such as cold sores (herpes simplex). Symptoms of full-blown AIDS which can be treated in accordance with the present invention, include, for instance, diarrhea, nausea and vomiting, thrush and mouth sores, persistent, recurrent vaginal infections and cervical cancer, persistent generalized lymphadenopathy (PGL), severe skin infections, warts and ringworm, respiratory infections, pneumonia, especially Pneumocystis carinii pneumonia (PCP), herpes zoster (or shingles), nervous system problems, such as pains, numbness or “pins and needles” in the hands and feet, neurological abnormalities, Kaposi's sarcoma, lymphoma, tuberculosis, and other opportunistic infections. Beneficial effects of the miRNAs or polynucleotides encoding said miRNAs include, for example, preventing or delaying initial infection of an individual exposed to HIV; reducing viral burden in an individual infected with HIV; prolonging the asymptomatic phase of HIV infection; maintaining low viral loads in HIV infected patients whose virus levels have been lowered via anti-retroviral therapy (ART); increasing levels of CD4 T cells or lessening the decrease in CD4 T cells, both HIV-1 specific and non-specific, in drug naive patients and in patients treated with ART, increasing overall health or quality of life in an individual with AIDS; and prolonging life expectancy of an individual with AIDS. A clinician can compare the effect of immunization with the patient's condition prior to treatment, or with the expected condition of an untreated patient, to determine whether the treatment is effective in inhibiting AIDS.

In another embodiment, the invention relates to a method for the treatment of a disease caused by HIV infection in a subject in need thereof comprising the administration to said subject of a microbicide of the invention.

Suitable routes of administration of the microbicides according to the invention have been described above in detail in the context of the medical uses of the miRNAs and polynucleotides encoding thereof and are equally applicable herein.

The microbicides of the invention may be administered using any suitable route including, but not limited to, by intramuscular, subcutaneous or intravenous injection, and oral, nasal, or anal route. See Banga A, “Parenteral Controlled Delivery of Therapeutic Peptides and Proteins,” in Therapeutic Peptides and Proteins (Technomic Publishing Co., Inc., Lancaster, Pa., US, 1995). In a preferred embodiment, the composition or kit-of-parts is administered topically. The medicament for the combined administration of a miRNA or polynucleotide encoding said miRNA and the anti-HIV agent may be prepared as a single dosing form or in separate dosing forms.

Microbicide compositions can be formulated in unit dosage form, suitable for individual administration of precise dosages. The dosage may be in the form of a single bolus or multiple doses, to be carried out for example, daily through certain period of time. The microbicide compositions can be administered whenever the effect (e.g. decreased signs, symptom, or laboratory results of HIV-1 infection) is desired. Generally, the dose is sufficient to treat or ameliorate symptoms or signs of disease without producing unacceptable toxicity to the subject. Systemic or local administration can be utilized. Further, the amount of the microbicide of the invention in a dosage form must be such to achieve an effective local anal, oral or vaginal concentration, that is to say, the compound(s) of the invention must be present at a level sufficient to originate a microbicide effect upon administration.

4. miRNA Signature-Specific Microarrays, Oligonucleotide Primers, and Kits

Following the identification of a miRNA signature according to the methods of the present invention, microarrays can be constructed which contain oligonucleotide probes that specifically recognize the miRNAs identified as comprising the miRNA signature. Thus, in another aspect, the invention relates to a kit comprising reagents adequate for the determination of the expression levels of one or more miRNAs selected from the group consisting of miR-27a, miR-27b, miR-29b, miR-221 miR-155, miR-146a, miR-484, miR-339, miR-186, miR-106a, miR-17, miR-590, miR-374b, miR-140-Sp, miR-16, miR-454, miR-200c, miR-146b, miR-200b, miR-422a, miR-125a-Sp, miR-191 and miR-197. In a preferred embodiment, the kit comprises reagents for the determination of the expression levels of the miRNAs selected from the group consisting of the miR-27a, miR-27b, miR-29b and miR-221.

Such microarrays are useful in the diagnostic, therapeutic, and screening assays described herein. Microarrays containing probes recognizing the subset of miRNAs comprising an miRNA signature have the advantages of being cost-effective to produce and simpler to analyze, as they do not include extraneous probes that are not relevant to the detection of a virus, the replication stage of a virus, or the disease or pathological state of a subject infected with a virus. In one embodiment, such a microarray also contains oligonucleotide probes specific for RNAs suitable for normalization purposes. Such probes are well known in the art, and include, for example, probes recognizing 18S ribosomal RNA, mRNA encoding GAPDH, and mRNA encoding beta-actin.

In a preferred embodiment, the reagents adequate for the determination of the expression levels of said one or more miRNAs comprise at least 50% of the total amount of reagents forming the kit.

In further embodiments, the reagents adequate for the determination of the expression levels of one or more miRNAs comprise at least 55% at least 60%, at least 65%, at least 70%, at least 75%, at least 80%, at least 90%, at least 95%, at least 96%, at least 97%, at least 98% or at least 99% of the total amount of reagents forming the kit.

In a related aspect, the present invention provides a set of one or more pairs of oligonucleotide primers designed to specifically amplify the miRNAs of a miRNA signature identified using the methods of the invention in a reverse-transcriptase polymerase chain reaction assay, preferably a real-time quantitative reverse-transcriptase polymerase chain reaction assay. Quantitative reverse-transcriptase polymerase chain reaction can provide a cost-effective alternative to microarray analysis when the number of miRNAs identified as part of a miRNA signature is relatively small.

The present invention further provides a kit containing a microarray that has miRNA-specific probe oligonucleotides which specifically recognize miRNAs that were identified as part of a miRNA signature using methods of the invention. The microarrays contained in the kit may also include probe oligonucleotides which specifically recognize RNAs suitable for normalization purposes. In certain embodiments, the kit further contains a control sample. This sample can be derived from a control or normal cell, tissue, or subject (i.e. one that exhibits normal traits). In an alternative aspect, the present invention provides a kit containing a set of one or more pairs of oligonucleotide primers designed to specifically amplify the miRNAs of a miRNA signature identified using the methods of the invention in a reverse-transcriptase polymerase chain reaction assay, preferably a real-time quantitative reverse-transcriptase polymerase chain reaction assay. In additional embodiments, the kits of the invention contain instructions for their use.

According to the present invention, a reagent which allows determining the expression level of a gene comprises: i) probes capable of specifically hybridizing with the mRNAs encoded by the gene and ii) compounds that bind specifically to the proteins encoded by the gene. In the latter case, the use of antibodies is preferred. Alternatively, aptamers may be utilized.

In a particular embodiment, the reagents of the kit of the invention are nucleic acids capable of detecting specifically the mRNA level of the genes mentioned above or the level of proteins encoded by one or more of the genes mentioned above. Nucleic acids capable of specifically hybridizing with the genes mentioned above can be one or more pairs of primer oligonucleotides for the specific amplification of fragments of the mRNAs (or of their corresponding cDNAs) of said genes. In a preferred embodiment, the kit of the invention comprises a probe which can specifically hybridize to the genes mentioned above. The hybridization reactions can be performed according to protocols known in the art. See Ausubel F, et al., Eds, Short Protocols in Molecular Biology, 4th Ed. (John Wiley and Sons, Inc., New York, N.Y., US, 1995).

This invention is further illustrated by the following examples which should not be construed as limiting. The contents of all references, patents and published patent applications cited throughout this application are incorporated herein in their entirety by reference.

### General Procedures

1. Research Subjects and Sample Processing

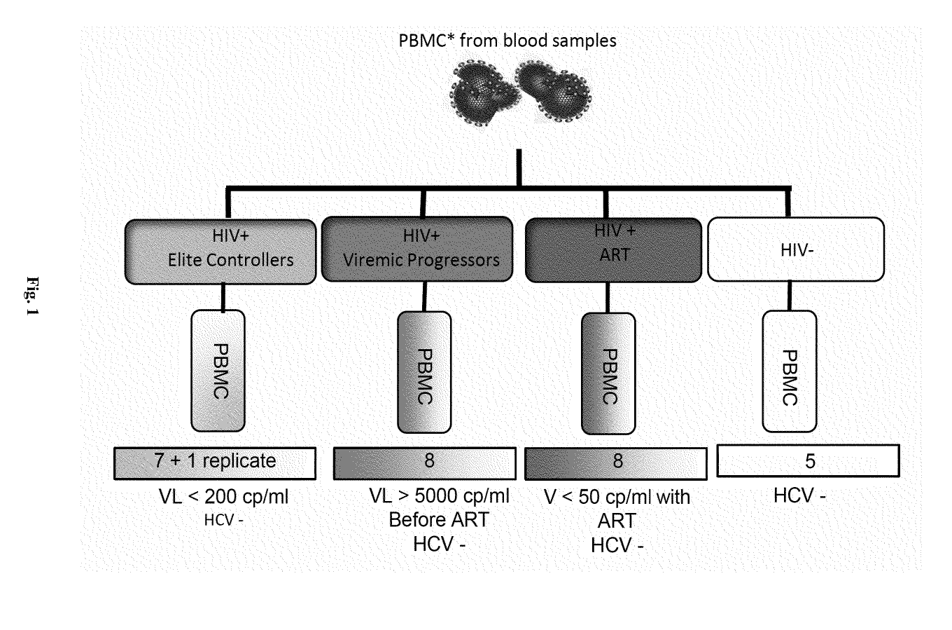

PBMC from four cohorts were taken from: elite controllers (EC) (n=8, viral load <50 cp/mL for more than 1 years, 3 titrations per year and CD4+ cells>450 cells/μL), viremic progressors (VP) before HAART treatment (n=8, mean plasma viral load=15,000 cp/mL, ART HIV infected patients (ART) (n=8, viral load<200 cp/mL with treatment) and HIV negative individuals (HIV−) (n=5). See FIG. 1. The viral load (cp/mL) and expression levels of HLA-A, HLA-B and HLA-C for the different patients are provided in Table 1.

TABLE 1
Individual Characteristics.

| | VL ⁽¹⁾ | HLA-A | | HLA-B | | HLA-C | |
|---|---|---|---|---|---|---|---|
| 1EC | 49 | *0301 | *0301 | *0702 | *4001 | *0302 | *0702 |
| 2EC | 199 | *0301 | *2902 | *4102 | *5801 | *0802 | *1705 |
| 3EC | 49 | *0101 | *6802 | *3501 | *5802 | *0401 | *0401 |
| 4EC | 92 | *2301 | *2902 | *0702 | *8101 | *0702 | *1801 |
| 5EC | 49 | *0301 | *2402 | *1801 | *5002 | *0501 | *0602 |
| 6EC | 49 | *0201 | *2601 | *5101 | *5701 | *0701 | *1402 |
| 7EC | 50 | *02 | *3201 | *0702 | *3801 | ND | ND |
| 8VP | 27800 | *2402 | *2902 | *4403 | *5201 | *1502 | *1601 |
| 9VP | 67965 | *2902 | *6901 | *4403 | *5701 | *0602 | *1601 |
| 10VP | 160000 | *0101 | *0301 | *5601 | *5701 | ND | ND |
| 11VP | 39195 | *0201 | *2501 | *1501 | *1801 | *0304 | *1203 |
| 12VP | 161000 | *0301 | *2402 | *1801 | *5002 | *0501 | ND |
| 13VP | 5374 | *2501 | *6801 | *1801 | *5501 | *0303 | *1203 |
| 14VP | 3933 | *0201 | *3201 | *4901 | *7802 | *0602 | *1402 |
| 15VP | 834 | *0101 | *0201 | *0801 | *3501 | ND | ND |
| 16ART | <50 | *0201 | *0311 | *1401 | *4001 | *0304 | *0802 |
| 17ART | <50 | *0301 | *2402 | *1402 | *4401 | *0501 | *0805 |
| 18ART | <50 | *0101 | *11 | *5701 | *39 | ND | ND |
| 19ART | <50 | *0101 | *24 | *1517 | *44 | ND | ND |
| 20ART | <50 | *0101 | *3201 | *1402 | *1501 | *0303 | *0802 |
| 21ART | <50 | *3101 | *68 | *4403 | *51 | ND | ND |
| 22ART | <50 | *03 | ND | *08 | *35 | ND | ND |
| 23ART | <50 | *02 | *03 | *08 | *5301 | ND | ND |
| 24C | — | — | — | — | — | — | — |
| 25C | — | — | — | — | — | — | — |
| 26C | — | *02 | *29 | *07 | *45 | — | — |
| 27C | — | — | — | — | — | — | — |
| 28C | — | — | — | — | — | — | — |

⁽¹⁾ VL EC was <200 cp/ml at least one year and CD4 for EC was >500 cells

Extracted plasma blood mononuclear cells (PBMCs) were cultured in RPMI modified media and activated with phyto-hemagglutinin 1% during 48 hours. Cells pellets were obtained after centrifugation (x g, x mn) and washed twice in sterile PBS 1×. Total RNA including small RNAs were extracted with MirVana kit (Ambion®, Life Technologies Corp., Carlsbad, Calif., US) according to the manufacturer's instructions. All RNA samples were quantified using nanodrop spectrophotometer and qualified with Agilent 2100 bioanalyzer using total RNA nano chip. Only samples with a RNA integrity number (RIN)>7 were processed. 350 ng of total RNA were simultaneously reverse transcribed and preamplified according to the Megaplex pools protocol (Applied Biosystems®, Life Technologies Corp., Carlsbad, Calif., US). The pre-amplified products were processed on TaqMan® Array Human MicroRNA Set Cards v2.0 (Applied Biosystems®, Life Technologies Corp., Carlsbad, Calif., US) and quantitative PCR was run on ABI 7900 (Applied Biosystems®, Life Technologies Corp., Carlsbad, Calif., US) according to the manufacturer's instructions.

2. Statistical Analysis

For each sample Ct (cycle threshold) values were calculated using RealTime StatMiner® Software (Integromics S.L., Granada, ES) after importation of the quantification values from sds 2.4 software (Applied Biosystems®, Life Technologies Corp., Carlsbad, Calif., US). Each Ct was normalized by subtraction of the mean value of all the expressed miRNAs of the same sample (global mean normalization) according to data published before. See Mestdagh P, et al., Mol. Cell 2010; 40(5):762-773. miRNAs with a Ct value >35 were considered as non-expressed and changed to a Ct value of 40. miRNAs that were not expressed in more than 25% of each cohort were excluded from the analysis. Finally a set of 168 expressed miRNAs genes was selected for the analysis.

A non parametric Mann-Whitney U test was run in TMEV software V4.5 (TM4: a free, open-source system for microarray data management and analysis) with a estimated false discovery rate (FDR) of 5%, between the groups elite controllers (EC) and viremic progressors (VP). FDR is used to control type I errors using Benjamini-Hochberg correction test. See Saeed A, et al., Biotechniques 2003; 34(2):374-378.

Fold change expression relative to HIV negative group (HIV−) for the differentially expressed miRNAs was calculated by comparative deltaCt method. Mean Ct normalized expression values of each miRNAs was calculated for EC and VP group and subtracted to the mean normalized Ct value of the reference group (HIV −). Fold change of each group relative to reference group was then calculated according to the 2^{-ΔCT} formula.

### Results

The expression of the human set of miRNAs (MicroRNA Set Cards v2.0, Applied Biosystems®, Life Technologies Corp., Carlsbad, Calif., US,) was evaluated by quantitative PCR from PBMCs of 4 groups of individuals. See FIG. 1.

After adequate normalization steps a statistic analysis of significant differentially expressed human miRNAs was performed between elite controllers (EC) and viremic progressors (VP). Non parametric Mann-Whitney U test set to a false discovery rate (FDR) of 5% provided a list of 23 candidate miRNAs. See Table 2.

TABLE 2
Candidate miRNA comparing EC with VP

| | | adj. P (FDR)* |
|---|---|---|
| Detector-miRNA | p-value | (Benjamini/Hochberg) |
| hsa-miR-454 | 7.78E−04 | 0.034755975 |
| hsa-miR-590-5p | 0.001131326 | 0.034755975 |
| hsa-miR-146b-5p | 0.001629186 | 0.034755975 |
| hsa-miR-200c | 0.001629186 | 0.034755975 |
| hsa-miR-422a | 0.001629186 | 0.034755975 |
| hsa-miR-27b | 0.001629186 | 0.034755975 |
| hsa-miR-27a | 0.002322095 | 0.037153512 |
| hsa-miR-16 | 0.002322095 | 0.037153512 |
| hsa-miR-221 | 0.003275898 | 0.044963185 |
| hsa-miR-191 | 0.00457444 | 0.044963185 |
| hsa-miR-374b | 0.00457444 | 0.044963185 |
| hsa-miR-29b | 0.00457444 | 0.044963185 |
| hsa-miR-339-3p | 0.00457444 | 0.044963185 |
| hsa-miR-146a | 0.006322948 | 0.044963185 |
| hsa-miR-17 | 0.006322948 | 0.044963185 |
| hsa-miR-125a-5p | 0.006322948 | 0.044963185 |
| hsa-miR-106a | 0.006322948 | 0.044963185 |
| hsa-miR-200b | 0.006322948 | 0.044963185 |
| hsa-miR-484 | 0.008651542 | 0.04814771 |
| has-miR-155 | 0.008651542 | 0.04814771 |
| hsa-miR-140-5p | 0.008651542 | 0.04814771 |
| hsa-miR-197 | 0.008651542 | 0.04814771 |
| hsa-miR-186 | 0.008651542 | 0.04814771 |

*5% of False Discovery Range (FDR)

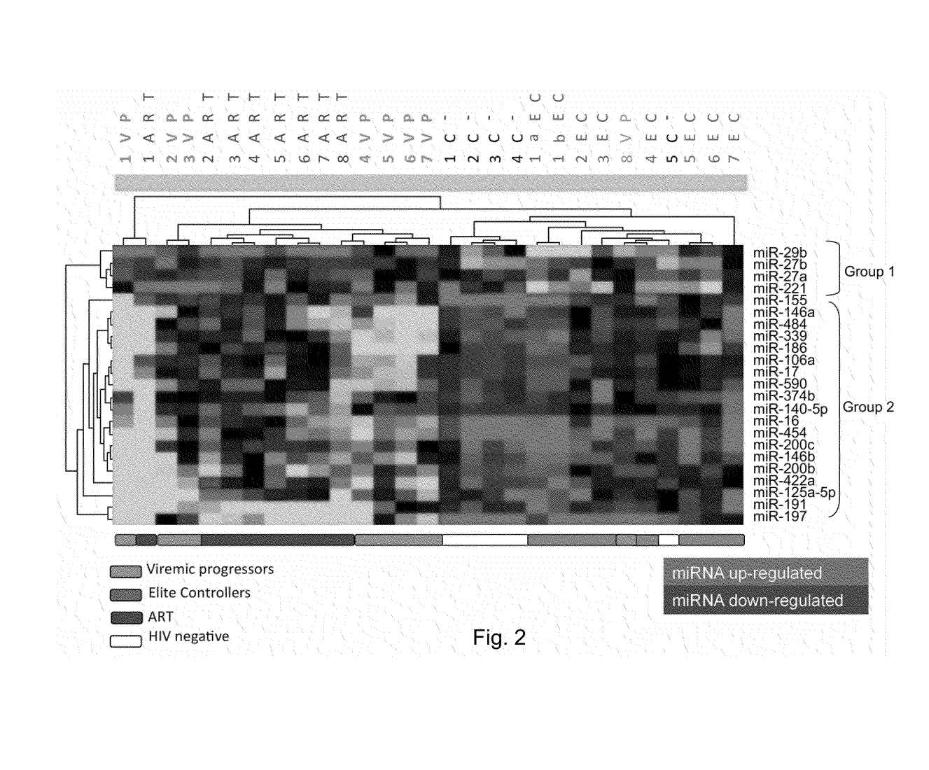

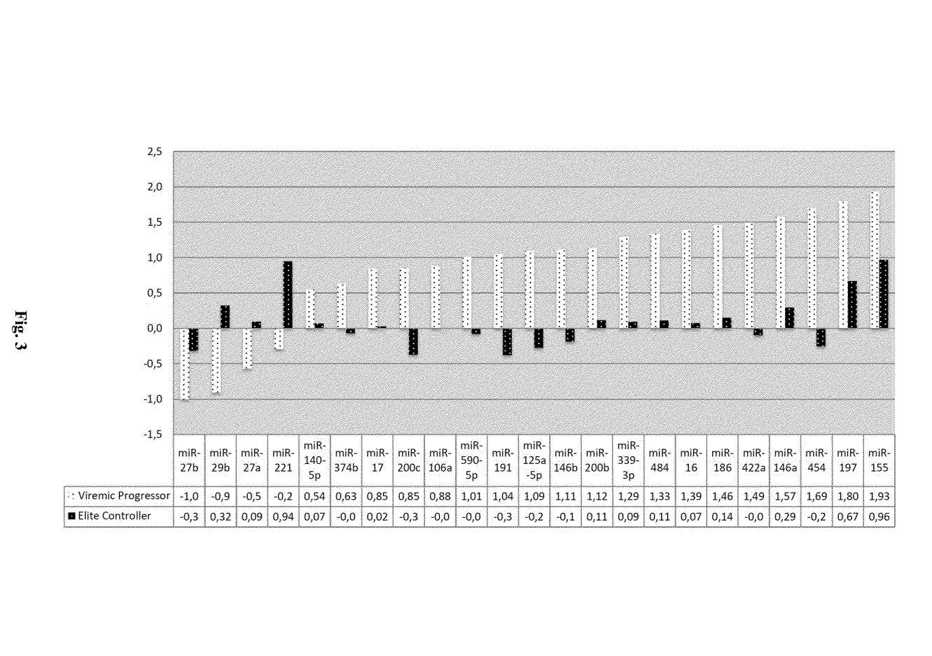

A hierarchical clustering (average linkage clustering constructed on Euclidian distances) of the candidates miRNAs of the 4 groups (EC; VP; ART; HIV Neg) of the study segregated the population in two separate blocks. See FIG. 2. The first block was more heterogeneous and included EC and HIV negative with no significant differences on miRNA expression, and the second block included VP and ART patients as a subgroup of this second block. One of the VP patients (8VP) is mixed between patients of the second block. Two miRNA expression patterns segregate between those two blocks; a set of 4 miRNAs are under expressed at the VP group and overexpressed in the EC (miR-221, 27a, 27b and 29b, group 1 on FIG. 2), whereas a set of 19 miRNAs (miR-155, miR-146a, miR-484, miR-339, miR-186, miR-106a, miR-17, miR-590, miR-374b, miR-140-Sp, miR-16, miR-454, miR-200c, miR-146b, miR-200b, miR-422a, miR-125a-Sp, miR-191 or miR-197) are overexpressed at the VP group and under expressed at the EC. See FIG. 2, group 2.

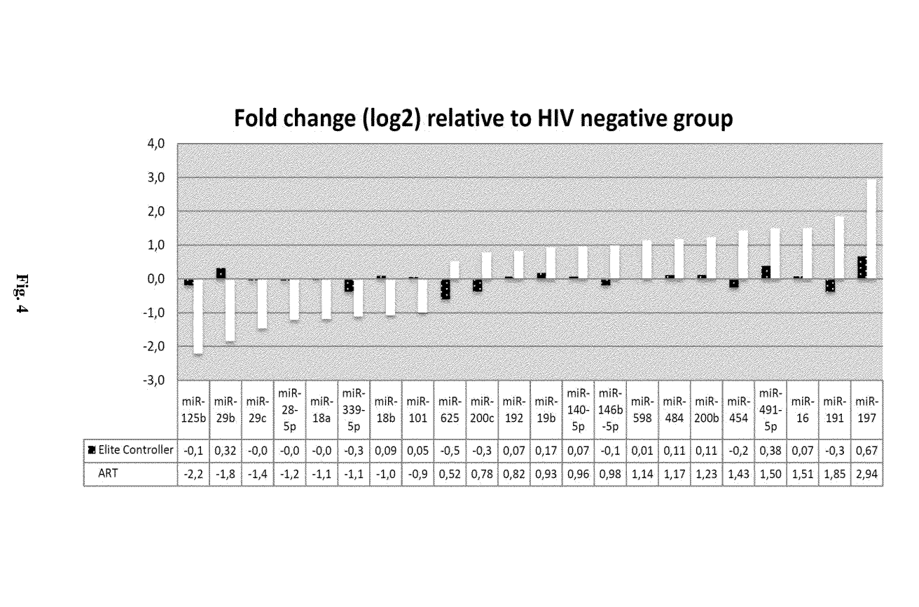

For selected miRNAs, results were expressed as the mean fold change of EC and VP relative to reference HIV negative group by the ΔΔCt method. The variation of this fold change is represented on a log 2 scale in FIG. 3A. On this graphical representation, close to “0” values are miRNAs that tend to have a similar expression compared to HIV negative group, whereas positive and negative values are miRNAs that are over or under expressed compared to HIV negative group.

Concerning the EC group, most of the miRNAs (miR-221, miR-197 and miR-155 excluded) values tend to have an expression value similar to HIV negative individuals. The fold change (mir-221, 197 and 155 excluded) ranged approximately from 1 to 1.3. miR-221 and miR-155 are the only ones to be clearly more up-regulated in this group (2 up-fold change).

Concerning the group of VP, 19 out of the 23 miRNAs are clearly up-regulated, h ranging from approximately 1.5 to 3.3 fold change. See FIG. 3A. Only 4 of them are down-regulated (from 1.2 to 2 down fold-change).

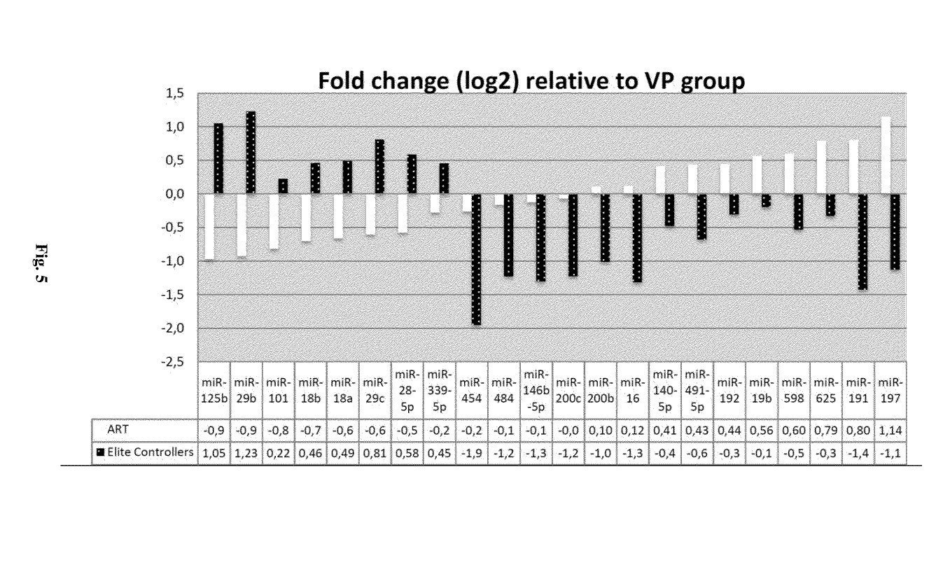

A comparison between EC and ART was performed in parallel and a total of 22 candidate miRNA were obtained. See Table 2. Again the fold change was calculated in EC and ART relative to HIV neg and VP, respectively. See FIGS. 4 and 5.

A more than 2 fold-change of miRNA expression of ART (14 up-regulated and 8 down-regulated) was observed relative to HIV neg group. See FIG. 4. However, after comparing EC and ART relative to VP, 10 miRNA (two up-regulated: miR-125 and miR-29b and eight down-regulated miR-454, miR-484, miR-146b, miR-200c, miR-200b, miR-16, miR-191, miR-197) showed more than 2 fold-change while other 12 miRNA (6 up-regulated: miR-101, miR-18a, miR-18b, miR-29c, miR-28-5p, miR-339-5p and 6 down-regulated: miR-140-5, miR-491-5p, miR-192, miR-19b, miR-598, miR-625) showed a fold-change lower than 2. See FIG. 5.

Analyzing the differential miRNA expression level, all possible comparisons between these groups were performed taking into account the possible miRNA signatures for each group related to viral load and immunogenetics. 10 out of 23 miRNA with a different expression level were observed when ECs were compared to VPs. The differential expression of the same 10 miRNA was observed when ECs were compared to the ART group. However, these 10 miRNA showed a different direction in expression, those overexpressed in the ECs versus VPs comparison were underexpressed in the ECs versus ARTs comparison and viceversa. No differential miRNA expression level was observed in the ECs versus HIV negs, the VPs versus ARTs, or the ARTs versus HIV negs comparisons. The HIV negs versus VPs and HIV negs versus ART comparisons yielded a total of 25 and 52 miRNAs that were differentially expressed, respectively. 17 out of the 25 miRNAs group were the same miRNAs as in the ECs versus VPs comparison.

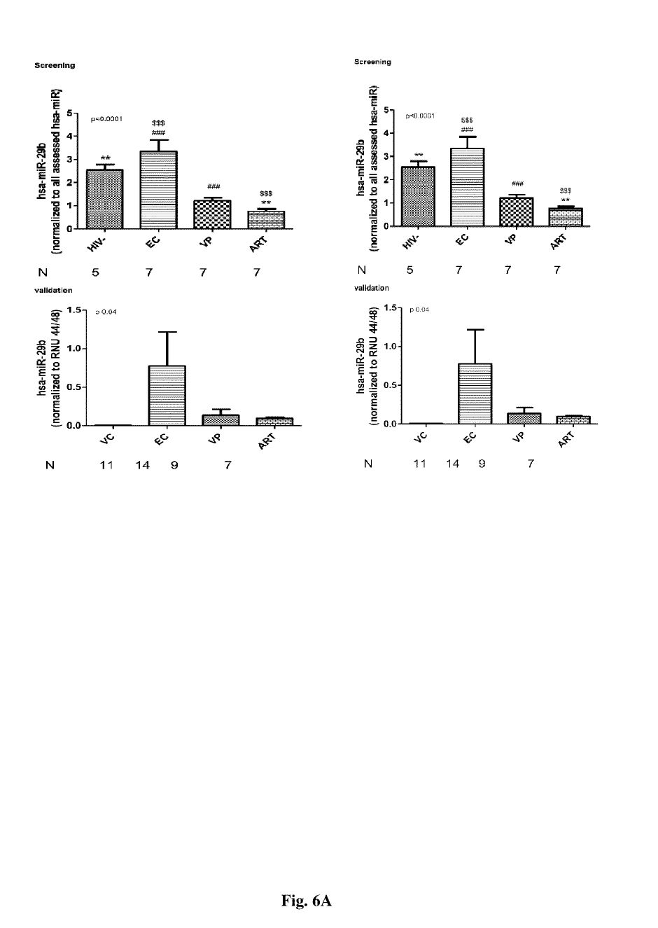

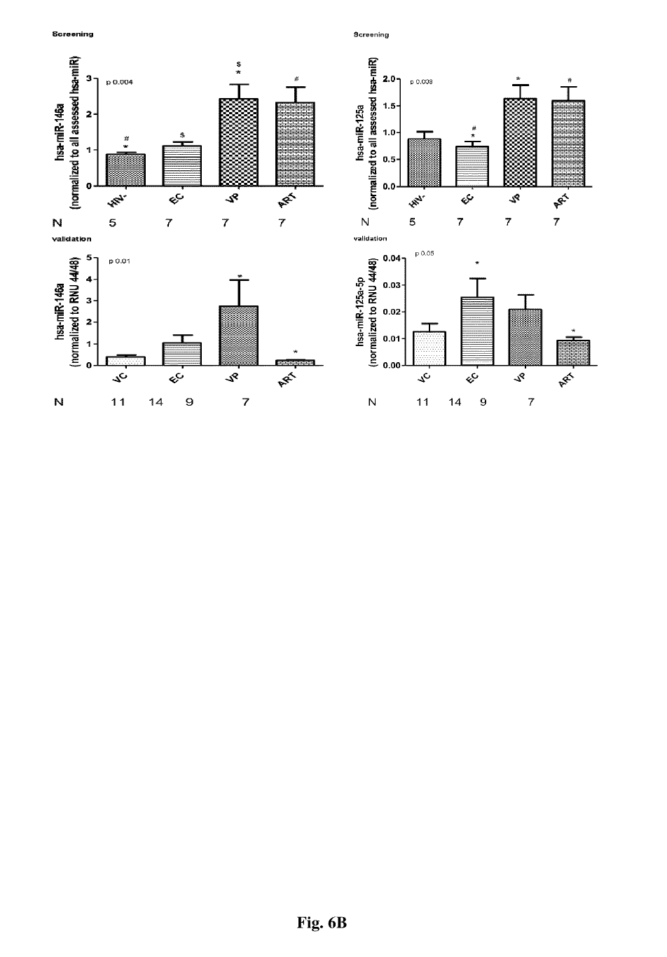

Afterwards, the results for miR-221, miR-29b, miR-125a, miR-146a using PBMC from new elite controllers (EC n=14), viremic progressors (VP n=9), treated HIV-1 individuals (ART n=7) were validated. After comparing the miRNA expression levels of these miRNAs between the initial “screening group” and the “validation group”, the same results were observed: miRNA-221 and miR-29b were upregulated, and miR-146a was downregulated compared to VPs. This downregulation was not observed for miRNA-125a. See FIGS. 6A and 6B.

1. A method for the identification of a controller HIV-Infected patient which comprises the determination of the level of one or more miRNAs selected from the group consisting of miR-27a, miR-27b, miR-29b, miR-221 miR-155, miR-146a, miR-484, miR-339, miR-186, miR-106a, miR-17, miR-590, miR-374b, miR-140-Sp, miR-16, miR-454, miR-200c, miR-146b, miR-200b, miR-422a, miR-125a-Sp, miR-191 and/or miR-197 in a sample from said patient, wherein increased expression level of one or more of the miR-27a, miR-27b, miR-29b and/or miR-221 miRNAs with respect to a reference value is indicative that the patient is a HIV controller or wherein decreased expression levels of one or more of miR-155, miR-146a, miR-484, miR-339, miR-186, miR-106a, miR-17, miR-590, miR-374b, miR-140-Sp, miR-16, miR-454, miR-200c, miR-146b, miR-200b, miR-422a, miR-125a-5p, miR-191 and/or miR-197 with respect to a reference value is indicative that the patient is a HIV controller.

2. A method according to claim 1 wherein the controller is an elite controller.

3. A method according to claim 1 wherein the controller is a viremic controller.

4. A method according to claim 1 wherein the sample is a PBMC preparation.

5. A method according to claim 1 wherein the reference value is the expression level of the miRNA in a sample from a viremic progressor or from a HIV-1 infected patient undergoing ART.

6. A method for the treatment or prevention of a disease caused by HIV in a subject in need thereof comprising administering to said subject a miRNA selected from the group consisting of miR-27a, miR-27b, miR-29b, miR-125, miR-146, miR-155 and miR-221.

7. A composition or kit-of-parts comprising at least one miRNA selected from the group consisting of miR-27a, miR-27b, miR-29b, miR-125, miR-146, miR-155 and miR-221 or a polynucleotide encoding said miRNA.

8. A composition or kit-of-parts according to claim 7 further comprising an anti-HIV agent.

9. (canceled)

10. A method for the treatment of a disease caused by HIV infection in a subject in need thereof comprising administering to said subject a composition or kit-of-parts according to claim 7.

11. A kit-of-parts comprising reagents adequate for the determination of the expression levels of one or more miRNAs selected from the group consisting of miR-27a, miR-27b, miR-29b, miR-221 miR-155, miR-146a, miR-484, miR-339, miR-186, miR-106a, miR-17, miR-590, miR-374b, miR-140-Sp, miR-16, miR-454, miR-200c, miR-146b, miR-200b, miR-422a, miR-125a-Sp, miR-191 and miR-197.

12. A kit-of-parts according to claim 11 wherein the reagents adequate for the determination of the expression levels of said one or more miRNAs are oligonucleotides that hybridize specifically to said one or more miRNAs.

13. A method according to claim 10 further comprising administering an anti-HIV agent to said subject.


SEQUENCE LISTING (23 sequences)

SEQ ID NO: 1 - RNA, 23 nt, H.sapiens
uagugcaauauugcuuauagggu

SEQ ID NO: 2 - RNA, 22 nt, H.sapiens
gagcuuauucauaaaagugcag

SEQ ID NO: 3 - RNA, 22 nt, H.sapiens
ugagaacugaauuccauaggcu

SEQ ID NO: 4 - RNA, 23 nt, H.sapiens
uaauacugccggguaaugaugga

SEQ ID NO: 5 - RNA, 22 nt, H.sapiens
acuggacuuagggucagaaggc

SEQ ID NO: 6 - RNA, 21 nt, H.sapiens
uucacaguggcuaaguucugc

SEQ ID NO: 7 - RNA, 21 nt, H.sapiens
uucacaguggcuaaguuccgc

SEQ ID NO: 8 - RNA, 22 nt, H.sapiens
uagcagcacguaaauauuggcg

SEQ ID NO: 9 - RNA, 23 nt, H.sapiens
agcuacauugucugcuggguuuc

SEQ ID NO: 10 - RNA, 23 nt, H.sapiens
caacggaaucccaaaagcagcug

SEQ ID NO: 11 - RNA, 22 nt, H.sapiens
auauaauacaaccugcuaagug

SEQ ID NO: 12 - RNA, 23 nt, H.sapiens
uagcaccauuugaaaucaguguu

SEQ ID NO: 13 - RNA, 23 nt, H.sapiens
ucccuguccuccaggagcucacg

SEQ ID NO: 14 - RNA, 22 nt, H.sapiens
ugagaacugaauuccauggguu

SEQ ID NO: 15 - RNA, 23 nt, H.sapiens
caaagugcuuacagugcagguag

SEQ ID NO: 16 - RNA, 24 nt, H.sapiens
ucccugagacccuuuaaccuguga

SEQ ID NO: 17 - RNA, 23 nt, H.sapiens
aaaagugcuuacagugcagguag

SEQ ID NO: 18 - RNA, 22 nt, H.sapiens
uaauacugccugguaaugauga

SEQ ID NO: 19 - RNA, 22 nt, H.sapiens
ucaggcucaguccccucccgau

SEQ ID NO: 20 - RNA, 23 nt, H.sapiens
uuaaugcuaaucgugauaggggu

SEQ ID NO: 21 - RNA, 22 nt, H.sapiens
cagugguuuuacccuaugguag

SEQ ID NO: 22 - RNA, 22 nt, H.sapiens
uucaccaccuucuccacccagc

SEQ ID NO: 23 - RNA, 22 nt, H.sapiens
caaagaauucuccuuuugggcu